(19) United States
(12) Patent Application Publication
(10) Pub. No.: US 2004/0044405 A1
(43) Pub. Date: Mar. 4, 2004

(54) Vascular stent or graft coated or impregnated with protein tyrosine kinase inhibitors and method of using same

(76) Inventors: Matthew R. Wolff, Middleton, WI (US); Scott William Stoker, Evansville, WI (US); Michael Orrin Griffin JR., San Diego, CA (US)

Correspondence Address: DEWITT ROSS & STEVENS S.C., 8000 EXCELSIOR DR, SUITE 401, MADISON, WI, 53717-1914, US

(21) Appl. No.: 10/280,576

(22) Filed: Oct. 25, 2002

Related U.S. Application Data
(60) Provisional application No. 60/343,732, filed on Oct. 25, 2001.

(51) Int. Cl. A61F 2/06

(52) U.S. Cl. 623/1.46

(57) ABSTRACT

Disclosed is a device, such as a cardiovascular stent, autologous venous/arterial graft, prosthetic venous/arterial graft, vascular catheter, or vascular shunt, for stenting a blood vessel, The device has coated thereon, adsorbed thereto, impregnated therein, or covalently or ionically bonded thereto an amount of a protein tyrosine kinase inhibitor. The protein tyrosine kinase inhibitor inhibits proliferation of vascular smooth muscle cells in an area within a blood vessel immediately adjacent to and/or proximal to the device, while simultaneously not inhibiting the proliferation of vascular intimal cells. A corresponding method of using the device to stent blood vessels is also disclosed.

### RELATED APPLICATION

Priority is hereby claimed to provisional application Serial No. 60/343,732, filed Oct. 25, 2001, which provisional application is incorporated herein by reference.

### FIELD OF THE INVENTION

The invention is directed to methods of selectively inhibiting the proliferation of vascular smooth muscle cells (VSMCs) following vascular injury or surgical interventions such as percutaneous revascularization, without inhibiting the prolifration of endothelial cells. Specifically, the invention is directed to the use of protein tyrosine kinase inhibitors, preferably those that inhibit the Bcr-Abl tyrosine kinase, and most preferably 4-{(4-methyl-1-piperazinyl)methyl}-N-{4-methyl-3-{{4-(3-pyrimidinyl}amino}-phenyl}benzamide methane sulfonate, coated onto vascular stents, native grafts, or prosthetic vascular grafts, to prevent the proliferation of VSMCs selectively, while not adversely affecting the proliferation of endothelial cells.

### BACKGROUND

Arteriosclerosis is a class of diseases characterized by the thickening and hardening of the arterial walls of blood vessels. Although all blood vessels are susceptible to this serious degenerative condition, the aorta and the coronary arteries serving the heart are most often affected. Arteriosclerosis is of profound clinical importance since it can increase the risk of heart attacks, myocardial infarctions, strokes, and aneurysms.

The traditional treatment for arteriosclerotic vessels currently includes vascular recanalization procedures for less-serious blockages and coronary bypass surgery for major blockages. Where possible, vascular recanalization is much preferred to coronary bypass because it is a far less invasive procedure. Vascular recanalization procedures involve using intravascular devices threaded through blood vessels to the obstructed site, including for example, percutaneous transluminal coronary balloon angioplasty (PTCA), also known as balloon angioplasty. Balloon angioplasty uses a catheter with a balloon tightly packed onto its tip. When the catheter reaches the obstruction, the balloon is inflated, and the atherosclerotic plaques are compressed against the vessel wall. A serious shortcoming of this and other intravascular procedures, however, is that in a significant number of treated individuals, some or all of the treated vessels restenose (that is, the vessels again narrow). This generally occurs in a relatively brief time period, roughly less than six months, after treatment. The restenosis is thought to be due in part to mechanical injury to the walls of the blood vessels caused by the balloon catheter or other intravascular device.

The walls of most blood vessels are composed of three distinct layers, or tunics, surrounding a central tubular opening, the vessel lumen. The innermost layer that lines the vessel lumen is called the tunica intima. The middle layer, the tunica media, consists mostly of circularly arranged smooth muscle cells and connective tissue fibers. In a non-injured vessel, the smooth muscle cells are generally not actively dividing. The outmost layer of the blood vessel wall, the tunica adventitia, is composed largely of collagen fibers that protect inner layers and gives the blood vessel structural integrity. Mechanical injury, resulting in damage to the tunica intima, initiates a cascade of events, including the release of chemicals such as platelet-derived growth factors (PDGF). This cascade prompts the migration and proliferation of vascular smooth muscle cells (VSMCs) at the site of injury. The accumulation of VSMCs at the site of injury narrows the diameter of the vessel lumen, thereby again putting the patient in danger of having a heart attack, stroke, etc.

Several methods for inhibiting smooth muscle cell proliferation following the use of an intravascular device have been reported in the patent literature. These include administering anti-proliferative agents such as cell cycle inhibitors and anti-coagulant agents (either by local or systemic delivery systems). Delivery of these agents systemically, however, has required dosages that cause unacceptable side-effects or are prohibitively expensive. Local delivery of agents, for example heparin, as described in U.S. Pat. No. 4,824,436, has proven ineffective in inhibiting restenosis due in part to inadequate residence time of the active agent at the site of injury. Cell cycle inhibitors such as taxol, which do not react covalently and therefore require prolonged residence time for effectiveness, suffer from similar problems. Moreover, prolonging residence times to increase the effectiveness of such treatments is also likely to present increased risks of toxicity.

Other methods reported for inhibiting VSMC proliferation involve local delivery of active agents contained in a sustained-release formulation. For example, U.S. Pat. No. 5,171,217 describes agents contained within a physiologically compatible, biodegradable polymeric microparticle. This formulation is delivered locally to the site of injury such that the agents are released from the arterial wall for 72 hours or more. In contrast, U.S. Pat. No. 6,281,225 describes the local, but non-sustained-release administration of DNA alkylating agents to prevent VSMC proliferation.

Another method for inhibiting smooth muscle cell proliferation involves administering photochemically-activated agents by local delivery systems. For example, U.S. Pat. No. 5,354,774 describes locally delivering 8-methoxypsoralen to the site of injury and then activating a photodynamic reaction using a visible light source.

Yet another approach to prevent proliferation of VSMCs is the use of radiation-emitting catheters or guide wires. These radioactive devices cause damage to nucleic acid, thus inhibiting replication and thereby inhibiting smooth muscle cell proliferation.

All of the above-described methods suffer from certain drawbacks. For example, sustained release formulations require an added level of complexity, namely incorporation of the agent on or within a sustained release formulation. Photodynamic therapy requires both local delivery of the photo-active agent and the use of a complex intravascular light source. Delivery of a radiation dose requires the presence of a radiologist and presents exposure hazards to the attending personnel, as well as material storage, handling, and disposal complications.

Treated coronary stents now on the market or in clinical trials also suffer from the distinct drawback that they inhibit the proliferation of endothelial cells. This contributes to thrombosis in the vicinity of deployed stent. Thrombosis has been observed in human clinical trials when using stents coated with either taxol or rapamycin. To prevent such thrombosis, the clinical patients have had to undergo a two- to three-month duration anti-coagulation treatment.

A need therefore exists for safe, simple, and straightforward method for inhibiting VSMC proliferation at a site of injury following vascular recanalization procedures or other vascular injury, without inhibiting the proliferation of endothelial cells. The ideal solution should be non-radioactive and require little or no retraining of medical personnel to implement.

Cellular signaling has become a major research theme in biology and medicine over the past twenty years. The complex pathways and protein components in signal transduction are emerging only slowly, but with increasing clarity. Over the last 15 years, the protein tyrosine kinases have been identified as key players in cellular regulation. They are involved in immune, endocrine, and nervous system physiology and pathology and thought to be important in the development of many cancers, most notably chronic myeloid leukemia. As such they serve as drug targets for many different diseases. A host of protein tyrosine kinases are known in the art. The attached Sequence List includes a non-exclusive sampling of the amino acid sequences of a number of such kinases.

As used herein, the term protein tyrosine kinases (PTKs) refers to any and all enzymes falling within the enzyme classification EC 2.1.7.112, without limitation. See the Sequence List, attached hereto, for various examples of PTKs. These enzymes catalyze the transfer of the gamma-phosphoryl group from ATP to the tyrosine hydroxyl moiety of a protein substrate. This family of kinases shares amino acid sequence homology with the serine/threonine kinase family. Although the number of tyrosine kinases being discovered is growing exponentially, molecular details pertaining to their substrate recognition, catalytic mechanism, and intra- and intermolecular regulation are still being elucidated.

As described in full below, the present inventors have found that inhibiting the action of PTKs selectively inhibits the proliferation of VSMCs.

### BRIEF DESCRIPTION OF THE DRAWINGS

FIG. 1 is a graph depicting porcine coronary vascular smooth muscle cell proliferation following stimulation with platelet-derived growth factor (PDGF) in the presence of increasing concentrations of STI-571.

FIG. 2 is a graph depicting porcine aortic endothelial cell proliferation following stimulation with vascular endothelial growth factor (VEGF) in the presence of increasing concentrations of STI-571.

FIG. 3 is a graph depicting inhibition of proliferation of human coronary artery vascular smooth muscle cells (hCASMC) by increasing concentrations of STI-571 (“Glivec”). Cells were counted after stimulation with 10% fetal bovine serum (FBS) for 48 hours, and data for each experiment was normalized to positive control wells containing FBS and no STI-571 (“Glivec”). Each point represents 18 to 21 wells from eight separate experiments, and is presented as the mean +/− the standard deviation.

FIG. 4 is a graph depicting inhibition of DNA synthesis in human coronary artery vascular smooth muscle cells (HCAVSMC) by STI-571 (“Glivec”). DNA synthesis was assayed by incorporation of BrdU after stimulation of coronary artery vascular smooth muscle cells by 10% FBS for 48 hours in the presence or absence (positive control) of STI-571 (“Glivec”). Data points represent 14 to 28 wells from two separate experiments, and are presented as the mean +/− the standard deviation.

FIG. 5 is a graph depicting inhibition of migration of human coronary artery vascular smooth muscle cells in response to STI-571 (“Glivec”). Migration was assayed by counting cells that migrated through a porous membrane (20 μm diameter pores) in 24 hours in response to stimulation with platelet-derived growth factor (PDGF-pp). Data bars represent six membranes, and are presented as means normalized to control membranes (no STI-571) +/− the standard deviation.

FIG. 6 is a graph depicting the lack of any effect of STI-571 (“Glivec”) on the proliferation of human coronary artery endothelial cells (hCAEC).

### DETAILED DESCRIPTION OF THE INVENTION

Abbreviations and Definitions:

The following abbreviations and definitions are used throughout the specification and claims. Terms not specifically defined herein have their normal and accepted meaning within the field of cardiovascular medicine and/or physiology.

“BrdU”=5-bromo-2′-deoxy-uridine triphosphate.

“DME”=Dulbecco's modified Eagle's media.

“FBS”=fetal bovine serum.

“JAK-2”=Janus-activated tyrosine kinases.

“MAPK”=mitogen-activated protein kinases.

“PDGF”=platelet-derived growth factor.

“Pharmaceutically-suitable salt”=any acid or base addition salt whose counter-ions are non-toxic to the patient in pharmaceutical doses of the salts, so that the beneficial inhibitory effects inherent in the free base or free acid PTK inhibitor are not vitiated by side effects ascribable to the counter-ions. A host of pharmaceutically-suitable salts are well known in the pharmaceutical field. For active ingredients that are bases, all acid addition salts are useful as sources of the free base form even if the particular salt, per se, is desired only as an intermediate product as, for example, when the salt is formed only for purposes of purification, and identification, or when it is used as intermediate in preparing a pharmaceutically-suitable salt by ion exchange procedures. Pharmaceutically-suitable acid addition salts include, without limitation, those derived from mineral acids and organic acids, explicitly including hydrohalides, e.g., hydrochlorides and hydrobromides, sulphates, phosphates, nitrates, sulphamates, acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates, quinates, and the like. In analogous fashion, for active ingredients that are acids, pharmaceutically-suitable base addition salts may be used. Base addition salts include, without limitation, those derived from alkali or alkaline earth metal bases or conventional organic bases, such as triethylamine, pyridine, piperidine, morpholine, N-methylmorpholine, and the like.

“PTCA”=percutaneous transluminal coronary balloon angioplasty.

“PTK”=protein tyrosine kinase; expressly defined herein as any and all enzymes falling within the enzyme classification EC 2.1.7.112, without limitation.

“PTK Inhibitor”=any compound or composition that selectively inhibits the catalytic activity of one or more protein tyrosine kinase inhibitors.

“STI-571”=4-{(4-methyl-1-piperazinyl)methyl}-N-{4-methyl-3-{{4-(3-pyrimidinyl}amino}-phenyl}benzamide and pharmaceutically-suitable salts thereof. The methane-sulphonate salt is preferred. This compound has been given the trivial generic name “imatinib.” As used herein, the term “STI-571” designates imatinib as either a free base or any pharmaceutically-suitable salt thereof, the mesylate salt being preferred. In the United States, it is marketed commercially by Novartis AG (Basel, Switzerland) under the registered trademark “Glivec” (U.S. T.M. Registration No. 2,478,196); it is also sold elsewhere around the world under the trademark “Gleevec.”

“VEGF”=vascular endothelial growth factor.

“VSMC”=vascular smooth muscle cells.

Overview:

Treating arteriosclerosis with intravascular devices, including for example, ablative procedures, balloon catheters, or vascular stents is becoming increasingly popular as technology related to intravascular devices continues to improve. Approximately 1 million balloon angioplasty procedures alone are performed on an annual basis globally. These procedures, however, have a major shortcoming. In a significant number of cases the treated vessels re-occlude, or restenose, by six months post-treatment which requires the individual to undergo additional treatment. “Restenosis” refers to the stage at which the vessel lumen has decreased in diameter by about 50% or more as compared to the diameter of the vessel lumnen immediately following a vascular recanalization procedure.

The pathogenesis of restenosis is not well understood. It is believed to be due, in part, to recoil of the wall of the treated vessel. Additionally, it is hypothesized that vascular recanalization procedures used to treat diseases, such as arteriosclerosis, can cause mechanical injury at the site of recanalization. Without being limited to any particular mechanism of action, it is hypothesized that once intimal rupture occurs in the blood vessel a number of events begin to take place including the migration of monocytes to the subendothelial layer of the intima and the release of mitogenic growth factors, including, for example, platelet-derived growth factor (PDGF), macrophage-derived growth factor (MDGF), and endothelial cell-derived growth factor (EDGF). These chemicals, and in particular PDGF, apparently play a role in inducing VSMC proliferation. This in turn produces substantial quantities of intercellular substances that accumulate within the vessel lumen, thereby narrowing its diameter.

A first embodiment of the present invention is therefore directed to a cardiovascular stent, autologous venous/arterial graft, prosthetic venous/arterial graft, vascular catheter or vascular shunt (collectively referred to herein as a “vascular device”) that is coated with one or more compounds that selectively inhibit the proliferation of VSMCs at the point immediately adjacent to and proximal to the point of vascular injury. Specifically, the invention comprises a vascular device that has coated thereon, adsorbed thereto, impregnated therein, or covalently or ionically bonded thereto an amount of a protein tyrosine kinase (PTK) inhibitor. It is preferred that the compound specifically inhibit the Bcr-Abl tyrosine kinase, the constituitive abnormal tyrosine kinase created by the Philadelphia chromosome abnormality found in chronic myeloid leukemia. Preferred PTK inhibitors for use in the invention are also those that specifically or non-specifically inhibit the activity of one or more PTKs selected from the group consisting of receptor tyrosine kinases for platelet-derived growth factor and stem cell factor (SCF), and c-Kit. The amount of the PTK used in conjunction with the vascular device is an amount sufficient to prevent or inhibit proliferation of vascular smooth muscle cells in an area within a blood vessel immediately adjacent to and/or proximal to the vascular device. In the preferred embodiment, the vascular device is coated with 4-{(4-methyl-1-piperazinyl)methyl}-N-{4-methyl-3-{{4-(3-pyrimidinyl}amino}-phenyl}benzamide and/or a pharmaceutically-suitable salt thereof (preferably the methane sulphonate salt).

A second embodiment of the invention is directed to a corresponding method for specifically preventing or inhibiting proliferation of VSMCs. Here, the method comprises coating, adsorbing, impregnating, or covalently or ionically bonding to the vascular device an amount of a PTK inhibitor; the amount being sufficient to prevent or inhibit proliferation of VSMCs in an area within a blood vessel immediately adjacent to and/or proximal to the vascular device when the device is deployed within the lumen of a blood vessel. The preferred PTK inhibitor is 4-{(4-methyl-1-piperazinyl)methyl}-N-{4-methyl-3-{{4-(3-pyrimidinyl}amino}-phenyl}benzamide and/or a pharmaceutically-suitable salt thereof.

A third embodiment of the invention is directed to a systemic method of preventing or inhibiting restenosis of blood vessels following vascular intervention. The method comprises systemically administering an amount of a PTK inhibitor (preferably orally), the amount administered being sufficient to prevent or inhibit proliferation of VSMCs in an area within a blood vessel immediately adjacent to and/or proximal to the area where the vascular intervention took place. Again, the preferred PTK inhibitor for use in this embodiment of the invention is 4-{(4-methyl-1-piperazinyl)methyl}-N-{4-methyl-3-{{4-(3-pyrimidinyl}amino}-phenyl}benzamide and/or a pharmaceutically-suitable salt thereof.

Compounds For Use in the Invention:

Any compound now known or discovered in the future that inhibits the action of PTKs can be used in the subject invention. Specific compounds whose anti-PTK activity has been documented, and thus can be used in the present invention, include (without limitation): pyridopyrimidines, phtalimides, chinolines, chinazolines, flavonoides, and benzothiazoles.

Among the most extensively studied PTK inhibitors are the tyrophostins and quinazoline derivatives. These compounds are currently under investigation as potential anti-cancer drugs. For example, tyrophostins and quinazoline have been shown to synergize with antibodies to EGFR and to established anti-cancer drugs like cisplatin to inhibit the growth of squamous cell carcinoma in vivo and to block the growth of human cancer cells over expressing HER2-ErbB2 (respectively). Tyrophostins are based on the benzylidenemalonitrile structure. Slight permutations in this structure have provided a range of potent inhibitors that selectively target EGFR, ErB-2 and v-Abl. Thus tyrophostins can be used alone or in combination with other PTK inhibitors to suppress VSMC proliferation into the lumen of blood vessels.

The quinazoline family of compounds includes the brominated quinazoline derivative, an early EGFR inhibitor that was found to be more than 3-fold more potent than any other tyrosine kinase inhibitor yet described (with an IC₅₀ of 29 pM). In addition, it has little affinity for PDGFR, FGFR, insulin receptor, the CSF receptor and Src, even at micromolar concentrations. Because of this extraordinary inhibitory activity and specificity, the quinazoline derivatives are a major focus of research aimed at developing kinase inhibitors as anti-cancer agents. Thus, these compounds can also be used in the present invention, either alone or in combination with other PTK inhibitors.

Another group of PTK inhibitory compounds, dianilinopthalimides, were rationally designed from the natural product PTK inhibitor staurosporine aglycon (see Appendix C). These compounds have been shown to be competitive inhibitors of ATP and to date more than 250 dianilinpthalimide derivatives have been synthesized and evaluated for their biological activity. The derivative CGP5211 has displayed a good amount of specificity towards EGFR (IC₅₀=3 mM), but also shows some inhibitory activity towards PKC. This observation led to the design of CGP53353 derivative, which showed lower specificity towards PKC isozymes. Thus, dianilinopthalimides can also be used as a PTK inhibitor in the present invention.

A large number of other compounds are known to be PTK inhibitors. These compounds, all of which can be used in the present invention, include bryostatins, defensins, genistein, H8, herbimycin A, tyrophostins, K-252a, lavendustin A, phorbol esters, staurosporines, and suramin.

The preferred PTK inhibitor for use in the present invention is 4-{(4-methyl-1-piperazinyl)methyl}-N-{4-methyl-3-{{4-(3-pyrimidinyl}amino}-phenyl}benzamide and pharmaceutically-suitable salts thereof (preferably the mesyl salt):

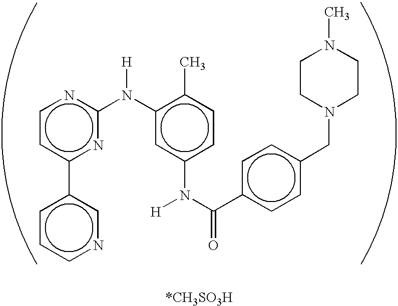

This compound, originally designated STI-571, is marketed commercially in the United States by Novartis under the trademark “Glivec.” It is approved by the U.S. Food and Drug Administration for the treatment of chronic myeloid leukemia. See EP 0 564 409 A and WO 99/03854.

Modes of Administration:

One embodiment of the invention is a method of preventing restenosis of blood vessels following a vascular injury or intervention by systemically administering one or more PTK inhibitors. The preferred route is orally. The PTK inhibitor may also be administered intravenously, intra-arterially, intramuscularly, percutaneously, parenterally, or rectally.

Specifically, systemic or topical administration is accomplished via a pharmaceutical composition comprising an active compound, i.e., a PTK inhibitor or a pharmaceutically-acceptable salt thereof, in combination with an acceptable carrier therefor and optionally in combination with other therapeutically-active ingredients or inactive accessory ingredients. The carrier must be pharmaceutically-acceptable in the sense of being compatible with the other ingredients of the formulation and not deleterious to the recipient. Suitable pharmaceutical compositions include those suitable for oral, topical (i.e. intra-lumen), rectal or parenteral (including subcutaneous, intramuscular and intravenous) administration.

The formulations may conveniently be presented in unit dosage form and may be prepared by any of the methods well known in the art of pharmacy. The term “unit dosage” or “unit dose” is denoted to mean a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

Formulations of the present invention suitable for oral administration may be presented as discrete units such as capsules, cachets, tablets, boluses or lozenges, each containing a predetermined amount of the active compound; as a powder or granules; or in liquid form, e.g., as an aqueous solution, suspension, syrup, elixir, emulsion, dispersion, or the like.

A tablet may be made by compression or molding, optionally with one or more accessory ingredients. Compressed tablets may be prepared by compressing in a suitable machine the active compound in a free-flowing form, e.g., a powder or granules, optionally mixed with accessory ingredients, e.g., binders, lubricants, inert diluents, surface active or dispersing agents. Molded tablets may be made by molding in a suitable machine a mixture of the powdered active compound with any suitable carrier.

Formulations suitable for parenteral administration conveniently comprise a sterile preparation of the active compound in, for example, water for injection, saline, a polyethylene glycol solution and the like, which is preferably isotonic with the blood of the recipient.

Useful formulations also comprise concentrated solutions or solids containing the PTK-inhibitory compound, which upon dilution with an appropriate solvent give a solution suitable for parenteral administration.

Preparations for topical or local applications comprise aerosol sprays, lotions, gels, ointments, suppositories etc., and pharmaceutically-acceptable vehicles therefore such as water, saline, lower aliphatic alcohols, polyglycerols such as glycerol, polyethylene glycerol, esters of fatty acids, oils and fats, silicones, and other conventional topical carriers. In topical formulations, the PTK inhibitors are preferably utilized at a concentration of from about 0.1% to 5.0% by weight.

Compositions suitable for rectal administration, comprise a suppository, preferably bullet-shaped, containing the active ingredient and pharmaceutically-acceptable vehicles therefore such as hard fat, hydrogenated cocoglyceride, polyethylene glycol and the like. In suppository formulations, the PTK inhibitors are preferably utilized at concentrations of from about 0.1% to 10% by weight.

Compositions suitable for rectal administration may also comprise a rectal enema unit containing the active ingredient and pharmaceutically-acceptable vehicles therefore such as 50% aqueous ethanol or an aqueous salt solution which is physiologically compatible with the rectum or colon. The rectal enema unit consists of an applicator tip protected by an inert cover, preferably comprised of polyethylene, lubricated with a lubricant such as white petrolatum and preferably protected by a one-way valve to prevent back-flow of the dispensed formula, and of sufficient length, preferably two inches, to be inserted into the colon via the anus. In rectal formulations, the PTK inhibitors are preferably utilized at concentrations of from about 5.0-10% by weight.

Useful formulations also comprise concentrated solutions or solids containing the active ingredient which upon dilution with an appropriate solvent, preferably saline, give a solution suitable for rectal administration. The rectal compositions include aqueous and non-aqueous formulations which may contain conventional adjuvants such as buffers, bacteriostats, sugars, thickening agents and the like. The compositions may be presented in rectal single dose or multi-dose containers, for example, rectal enema units.

Preparations for topical or local surgical applications for treating a blood vessel within its lumen comprise swabs or catheters suitable for such purposes. In both topical or local surgical applications, the sterile preparations of PTK inhibitor are preferably utilized at concentrations of from about 0.1% to 5.0% by weight applied to a dressing.

Compositions suitable for administration by inhalation include formulations wherein the active ingredient is a solid or liquid admixed in a micronized powder having a particle size in the range of about 5 microns or less to about 500 microns or liquid formulations in a suitable diluent. These formulations are designed for rapid inhalation through the oral passage from a conventional delivery systems such as inhalers, metered-dose inhalers, nebulizers, and the like. Suitable liquid nasal compositions include conventional nasal sprays, nasal drops and the like, of aqueous solutions of the active ingredient(s).

In addition to the aforementioned ingredients, the formulations of this invention may further include one or more optional accessory ingredient(s) utilized in the art of pharmaceutical formulations, i.e., diluents, buffers, flavoring agents, colorants, binders, surface active agents, thickeners, lubricants, suspending agents, preservatives (including antioxidants) and the like.

The amount of the PTK inhibitor required to be effective for inhibiting VSMC proliferation will, of course, vary with the individual mammal being treated and is ultimately at the discretion of the medical or veterinary practitioner. The factors to be considered include the condition being treated, the route of administration, the nature of the formulation, the mammal's body weight, surface area, age and general condition, and the particular PTK inhibitor to be administered. In general, a suitable effective dose is in the range of about 0.01 to about 500 mg/kg body weight per day of the selected PTK inhibitor. The total daily dose may be given as a single dose, multiple doses, e.g., two to six times per day, or by intravenous infusion for a selected duration. Dosages above or below the range cited above are within the scope of the present invention and may be administered to the individual patient if desired and necessary.

The PTK inhibitors may be administered prophylactically (in the preferred embodiment immediately post-surgery), chronically, or acutely.

Specifically addressing the preferred embodiment, Novartis sells STI-571 in capsules that provide the equivalent of 100 mg of the free base form of STI-571. When administered orally (the preferred route), the preferred amount of STI-571 for use in the present invention is from 100 to 800 mg daily, taken in from one to four equal doses. Considerably larger doses, up to 1,200 mg/m²/day, may also be given. Doses above 1,200 mg/m²/day are not recommended.

The methods of the present invention include the administration, by local delivery to a site of injury, of compounds that have the ability to inhibit PTK activity. Non-limiting examples of local delivery systems for use in the present invention include intravascular drug delivery catheters, wires, pharmacological stents and endoluminal paving.

In the preferred embodiment using local delivery, the compounds for use in the present invention are administered to the site of recanalization by direct intravascular deposition using intravascular catheters. Catheter systems for use in the present invention, include, for example, pressure-driven catheters, diffusion catheters and mechanical catheters. Pressure-driven catheter systems that can be used in the present invention include porous catheters; microporous catheters, for example, those made by Cordis Corporation; macroporous catheters; transport catheters, for example, those made by Cardiovascular Dynamics/Boston Scientific; channeled balloon catheters, for example, those made by Boston Scientific; and infusion sleeve catheters, for example, those made by LocalMed. See, for example, U.S. Pat. No. 5,279,565.

The PTK inhibitors may also be administered locally via diffusion-based catheter systems, including for example, double balloon, dispatch, hydrogel and coated stent catheters. The methods of the invention also include local administration of the compounds used in the methods of the present invention by mechanical device-based catheter systems, such as iontophoretic balloon catheters.

The compounds for use in the present invention may be administered by local delivery at a time proximal to the recanalization procedure or at a time after the recanalization procedure. The compounds for use in the invention may be delivered in a single dose or delivered in repeat doses.

The ability to deliver the PTK inhibitory compounds used in the present invention may be evaluated in vivo using known animal models, including the porcine coronary model described in the Examples. Thus, for example, a PTK inhibitor to be used in the methods of the present invention is administered by local delivery to a porcine at a site of vascular injury. The porcine is sacrificed and then examined by known cytological, histological, and other methods, including, for example, fluorescence microscopy.

Optimum conditions for delivery of the PTK inhibitory compounds for use in the methods of the invention may vary with the different local delivery systems used, as well as the properties and concentrations of the compounds used. Conditions may be optimized for inhibition of VSMC proliferation at the site of injury such that significant arterial blockage due to restenosis does not occur, as measured, for example, by the proliferative ability of the VSMCs, or by changes in the vascular resistance or lumen diameter. Conditions which may be optimized include, for example, the concentrations of the compounds, the delivery volume, the delivery rate, the depth of penetration of the vessel wall, the proximal inflation pressure, the amount and size of perforations and the fit of the drug delivery catheter balloon.

In a particularly preferred route of administration, the PTK inhibitory compound is coated or adsorbed onto a vascular stent, a prosthetic venous/arterial graft, or an autologous vascular graft. Alternatively, the PTK inhibitor may be impregnated therein, or covalently or ionically bonded thereto.

The preferred application of the PTK inhibitor to the stent, graft, or prosthesis is by conventional methods which are known in the art. These methods include, without limitation, dipping, steeping and spraying the article with the PTK inhibitor. Additional coating and impregnation techniques using pressure to force the coating into the substrate interstices are also contemplated. Multiple layers of the bio-active coating may be applied to the article. The stent, graft or prosthesis may first be coated with a polymeric coating to provide sustained release of the PTK inhibitor over a period of days, week, or months. Preferably, from about 1 to about 10 layers of the PTK inhibitory agent are applied to the surface of the stent, graft, or prosthesis.

Devices and Autologous Grafts According to the Invention:

As noted in the previous paragraph, one preferred route to administer the PTK inhibitory compounds is to adhere them onto a stent, autologous graft, or vascular prosthesis. In the present invention, any such vascular medical device may be used, including catheters, stents, sheets, tubes, balloons, and the like. The term “medical device” as used herein shall generically designate all such vascular medical devices, whether synthetic, semi-synthetic, or autologous tissue or material.

Preferably the medical device of the present invention is an implantable device such as a vascular graft, endoprosthesis or stent, that has been treated, coated, or otherwise manipulated to have coated on at least one surface a compound that inhibits PTK activity. For purposes of this invention, the term “vascular graft” is meant to include all endoprostheses which are generally introduced via catheter. In the preferred embodiment, the medical device is coated with STI-571. Other medical devices may also be coated, such as catheters which are minimally invasive. The vascular graft may include a hollow tubular body having an inner and an outer hydrophobic surface, the outer surface or both surfaces of which are coated with the PTK inhibitory compound.

Most preferably, the device of the present invention is a small caliber vascular stent or graft, made of metal or polymeric material (such as poly(tetrafluoroethylene)). This includes stents made of polymeric materials and coated with distinct materials, such as the polytetrafluoroethylene stent described in U.S. Pat. No. 6,306,165.

Vascular stents, the preferred medical device of the subject invention, are miniature mesh tubes that are implanted in the arteries to keep blocked portions open after angioplasty procedures. Working as scaffolding for the treated artery, stents are flexible yet quite strong, are generally easy (for a skilled physician) to deliver via catheter, and are readily seen on a fluoroscope. Stents are pre-mounted on balloon catheters which are used to deliver the stent to the treatment site and then expand the stent into place after the blockage is cleared.

Any stent now known or developed in the future can be coated with a PTK inhibitor according to the present invention. Perhaps the largest commercial supplier of vascular stents is Medtronic, 710 Medtronic Parkway, Minneapolis, Minn. Medtronic also has facilities located in Tolochenaz, Switzerland; Ontario, Canada; Causeway Bay, Hong Kong; and Gladesville, NSW, Australia. All of Medtronics stents, catheters, balloons, guide catheters, guidewires, and the like can be used in the present invention. Currently, Medtronic markets a very wide range of stents and other vascular medical devices under the “Discrete Technology,” “S7,” “S670,” “S660,” and “BeStent” trademarks.

Vascular stents are available from non-US-based manufacterers as well. For example, Biocompatibles Cardiovascular, of Farnham, United Kingdom, manufactures and sells a range of cardiovascular stents under the trademark “BiodivYsio.”

The PTK inhibitor can be adhered or coated onto the medical device, or it can be chemically bonded, either covalently or ionically to the medical device. The PTK inhibitor may be bonded directly to the medical device, or bonded via a spacer group or linker. For covalent attachment, it is preferred that a polymeric medical device, or a polymer-coated medical device be used and that the PTK inhibitor be covalently bonded to the medical device via a spacer group or linker having a chain length of from 1 to 250 atoms. For example, the spacer group may include an alkyls, alkylamines, oxygenated polyolefins, aliphatic polyesters, polyamino acids, polyamines, hydrophilic polysiloxanes, hydrophilic polysilazanes, hydrophilic acrylates, hydrophilic methacrylates, linear and lightly branched polysaccharides, and the like.

In yet another embodiment of the invention, there is provided a surface-modified implantable sheet material whose treated surface when exposed to the intimal layer of a blood vessel exhibits anti-VSMC proliferation activity over extended periods of time. This implantable sheet material includes a hydrophobic substrate material having adhered or bonded thereto a compound that inhibits PTK activity, the preferred compound being STI-571. The sheet can be formed into surgical mesh patches or tubes to repair vascular defects and injuries.

### EXAMPLES

The following Examples are included solely to provide a more thorough disclosure of the invention claimed herein. The Examples do not limit the invention in any fashion.

### Example 1

### Vascular Smooth Muscle Cell Proliferation

Porcine coronary VSMCs were grown to subconfluence in 96-well plates with DME media containing 10% FBS at 37° C. for 3 to 5 days. After synchronization in serum-free DME media for 48 hours, the cells were stimulated with PDGF (20 ng/mL) for 24 hours, in the presence of STI-571 (0.01 to 10 M). BrdU was added to the wells for the last 5 hours of the stimulation period. The cells were subsequently dried for 24 hours at 60° C., fixed and denatured, and BrdU incorporation was determined using a calorimetric assay (ELISA) sold commercially by Roche Molecular Biochemicals (catalog no. 1,647,229), following the manufacturer's protocol. See “Cell Proliferation ELISA, BrdU (Colorimetric) Instruction Manual,” Version 3, September 2000, available from Roche Molecular Biochemicals. Briefly, the BrdU ELISA is a calorimetric immunoassay for quantification of cell proliferation. It is based on the measurement of BrdU incorporation during DNA synthesis. The calorimetric approach is a non-radioactive alternative to the equivalent ³H-thymidine incorporation assay. See also Example 4.

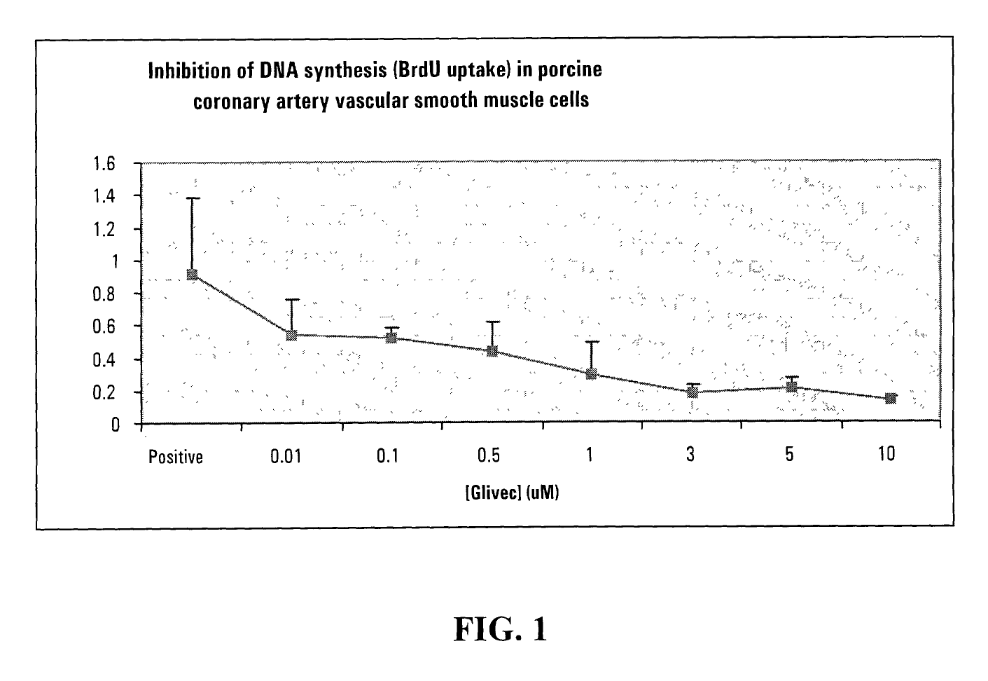

The results of this Example are presented graphically in FIG. 1. DNA synthesis was assayed by incorporation of BrdU (in the same fashion as described in Example 4) after stimulation of the cells with platelet-derived growth factor (PDGF-ββ, 20 ng/ml) for 48 in the presence or absence (positive control) of STI-571. Each data point represents 5 to 7 wells, and is expressed as he mean +/− the standard deviation.

As can be seen from the figure, administration of STI-571 inhibited the proliferation of VSMCs in a dose-dependent fashion. This Example demonstrates the utility of the present invention to inhibit the proliferation of VSMCs.

### Example 2

### Vascular Endothelial Cell Proliferation

Porcine aortic vascular endothelial cells were grown to subconfluence in 96-well plates with DME media containing 10% FBS at 37° C. for 3 to 5 days. After synchronization in serum-free DME media for 48 hours, the cells were stimulated with VEGF (20 ng/mL) for 24 hours, in the presence of STI-571 (0.01 to 10 M). BrdU was added to the wells for the last 5 hours of the stimulation period. The cells were subsequently dried for 24 hours at 60° C., fixed and denatured, and BrdU incorporation determined using the Roche ELISA described in Example 1.

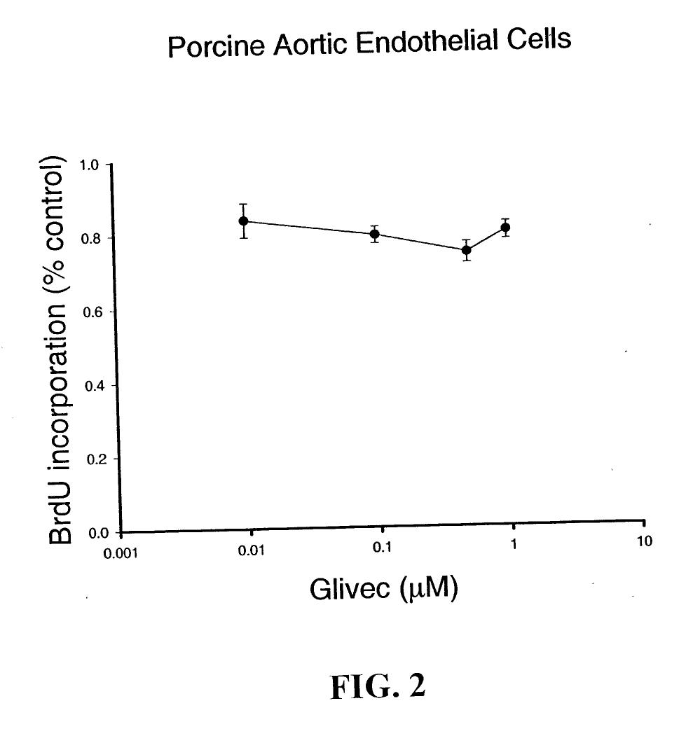

The results of this Example are presented graphically in FIG. 2. As can be seen from this figure, STI-571 had a very minimal inhibitory effect on the proliferation of aortic vascular endothelial cells.

Taken in conjunction with the results of Example 1, this Example demonstrates the utility of the present invention to inhibit the proliferation of VSMCs selectively, while not having an appreciable inhibitory affect on the proliferation of aortic vascular endothelial cells.

### Example 3

### Inhibition of Proliferation of Human Coronary Artery Vascular Smooth Muscle Cells by Increasing Concentrations of STI-571

This Example demonstrates that human coronary artery vascular smooth muscle cells are inhibited in a dose-dependent fashion by STI-571.

Cyropreserved human coronary artery vascular smooth muscle cells (CC-2583) were purchased commercially from Clonetics (now a wholly-owned subsidiary of Cambrex Bio Science Walkersville, Inc., Walkersville, Md.).

The cells were grown in canted-neck, filtered-cap, 25 cm² culture flasks, at an initial seed density of 2500 cells per cm². The cells were grown in “SmGM-2”-brand smooth muscle growth medium (Cambrex, used as delivered from the manufacturer) plus 10% FBS in a humidified 37° C., 5% CO₂ incubator. Media were changed initially after 24 hours, and then every 48 hrs subsequently. The cells were passed at approximately 80% confluency (˜4-6 days). The proliferation assays were performed in 24-well culture plates.

On day 5, growth media were replaced with test media (growth media+STI-571), growth media (positive control, media+FBS), and serum-free media (negative control, the “SmGM-2”-brand media without any added FBS).

Cells were counted manually trypsinizing the cells on day 7, with each condition (3 wells) pooled into one micro-centrifuge tube. The cells were spun at 1.5×g for 10 min. and then resuspended in 60 μl trypsin-neutralizing solution. The cells were then counted on a hemacytometer in quadruplicate.

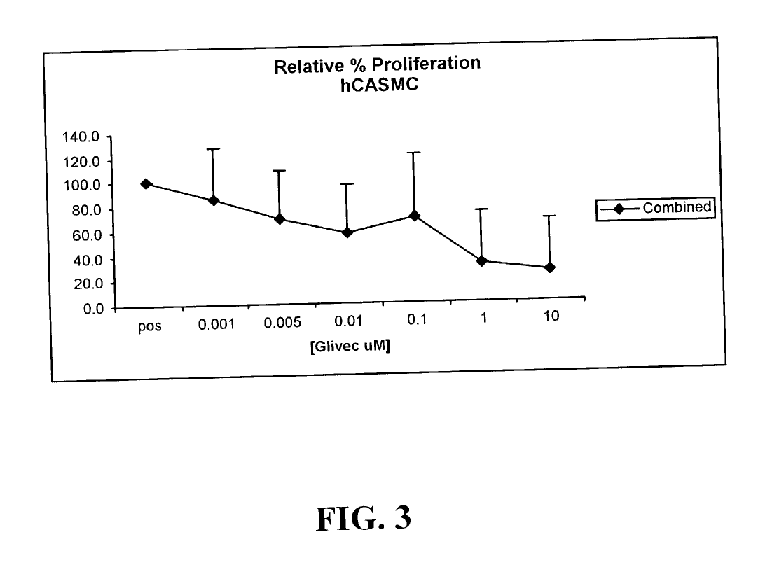

The results are shown in FIG. 3. In the figure, cells were counted after being stimulated with 10% FBS for 48 hours. The data for each experiment was normalized to positive control wells containing FBS and no STI-571. Each point represents 18 to 21 wells from eight separate experiments. The center of each data point is the mean at each concentration of STI-571, and the error bars are the standard deviation at each concentration level.

The significance of this graph is that it clearly indicates that STI-571 inhibits, in a dose-dependent fashion, the proliferation of human coronary artery vascular smooth muscle cells. Because these cells are responsible for restenosis, this graph demonstrates the effectiveness of the present invention for inhibiting such restenosis.

### Example 4

### Inhibition of DNA Synthesis in Human Coronary Artery Vascular Smooth Muscle Cells by STI-571

This example demonstrates that STI-571 inhibits DNA synthesis in human coronary artery vascular smooth muscle cells.

The same cells as described in Example 3 were used. Culture conditions and exposure to the various test concentrations of STI-571 were also the same as in Example 3.

DNA incorporation was measured using a commercially-available BrdU assay (Roche Molecular Biochemicals, catalog no. 1,647,229). The BrDu labeling solution was added on day 6, and the cells then allowed to incubate for another 24 hrs (through day 7). The label solution was then removed and the cells were dried at 60° C. for one hour. The cells were then fixed using “FixDenat” fixing solution for one hour at room temperature. The fixing solution was then removed and anti-BrdU antibody solution added to the cells. The cells were then incubated for 2 hr at 37° C.

The antibody solution was then removed substrate added to the wells. The plates were incubated at room temperature until sufficient color development occurred. The reactions were stopped by adding 1 M H₂SO₄ to the wells. The absorbance was then measured at 450 nm (reference, 690 nm).

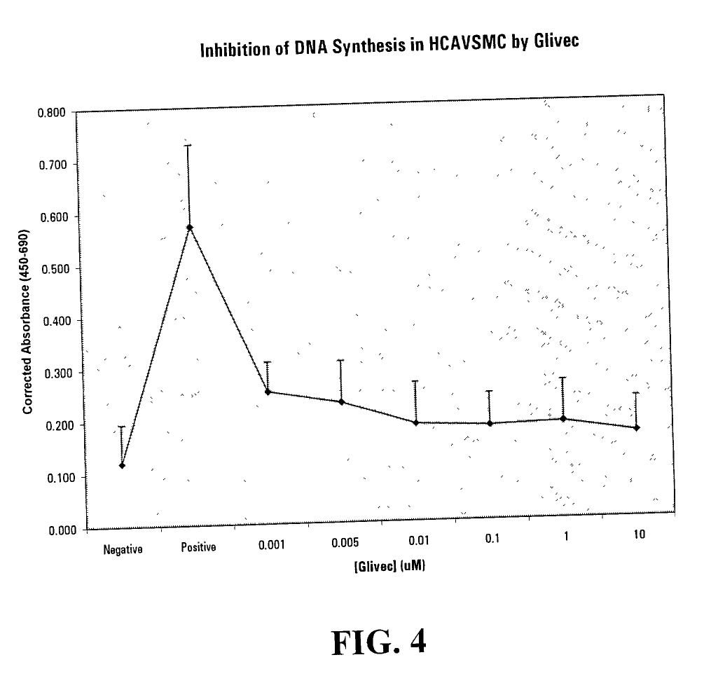

The results are shown in FIG. 4. Each data point represents 14 to 28 wells from two separate experiments, and are expressed as the means +/− the standard deviations. The significance of this Example is that it shows that STI-571 inhibits DNA synthesis in human coronary artery vascular smooth muscle cells. As in the previous Example, this is notable because these types of cells cause restenosis of stented vessels. By inhibiting the growth of such cells, restenosis is inhibited.

### Example 5

### Inhibition of Migration of Human Coronary Artery Vascular Smooth Muscle Cells by STI-571

This Example was performed to determine if STI-571 has any effect on the migration of human coronary vascular smooth muscle cells.

The cells described in Example 3 were used. The initial seed density was 4000 cells per filter (0.3 cm²) in test media with 1% BSA and 20 ng/ml PDGF-ββ. The cells were then incubated in a humidified environment at 37° C., 5% CO₂ for 24 hrs. The cells on the top side of the filter were then scraped away. The cells on bottom side of the filters were then fixed with ice-cold methanol for 10 min. The filters were rinsed with PBS and then stained with HarrisAE Hematoxylin stain for 5 min. and again rinsed with PBS.

The cells were then counted manually under high-power magnification (400×) in quadruplicate.

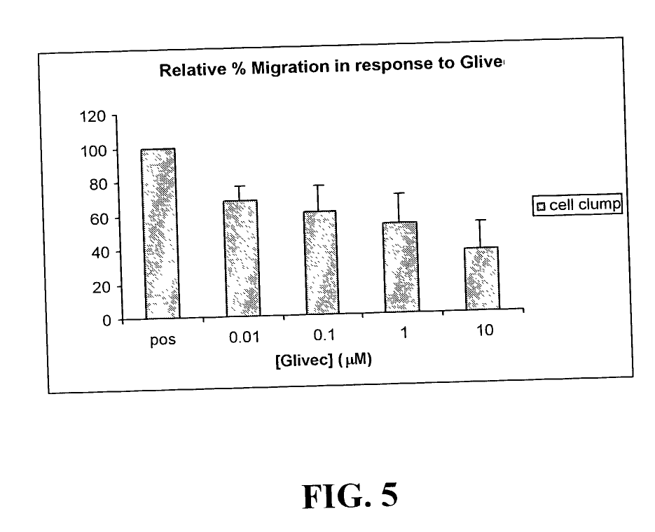

The results are shown in FIG. 5. Data bars represent 6 membranes, and the data are presented as means normalized to control membranes (no STI-571) +/− standard deviations.

The significance of this Example is that it demonstrates that STI-571 inhibits the migration of human coronary vascular smooth muscle cells in a dose-dependent fashion. Because migration of these cells is a major contributor to restenosis after deployment of a stent, this Example demonstrates that the present invention can be used to inhibit this migration and hence inhibit restenosis.

### Example 6

### Lack of Inhibitory Effect of STI-571 on Proliferation of Human Coronary Artery Endothelial Cells

This Example demonstrates that the growth of human coronary artery endothelial cells are not inhibited in any fashion by STI-571.

Cyropreserved human coronary artery endothelial cells were purchased commercially from Clonetics (now a wholly-owned subsidiary of Cambrex Bio Science Walkersville, Inc., Walkersville, Md.).

The cells were grown in canted-neck, filtered-cap, 25 cm² culture flasks, at an initial seed density of 2500 cells per cm². The cells were grown in “EGM-MV”-brand smooth muscle growth medium (Cambrex, used as delivered from the manufacturer) plus 10% FBS in a humidified 37° C., 5% CO₂ incubator. Media were changed initially after 24 hours, and then every 48 hrs subsequently. The cells were passed at approximately 80% confluency (˜4-6 days). The proliferation assays were performed in 24-well culture plates.

On day 5, growth media were replaced with test media (growth media +STI-571), growth media (positive control, media+FBS), and serum-free media (negative control, the “EGM-MV”-brand media without any added FBS).

Cells were counted manually trypsinizing the cells on day 7, with each condition (3 wells) pooled into one micro-centrifuge tube. The cells were spun at 1.5×g for 10 min. and then resuspended in 60 μl trypsin-neutralizing solution. The cells were then counted on a hemacytometer in quadruplicate.

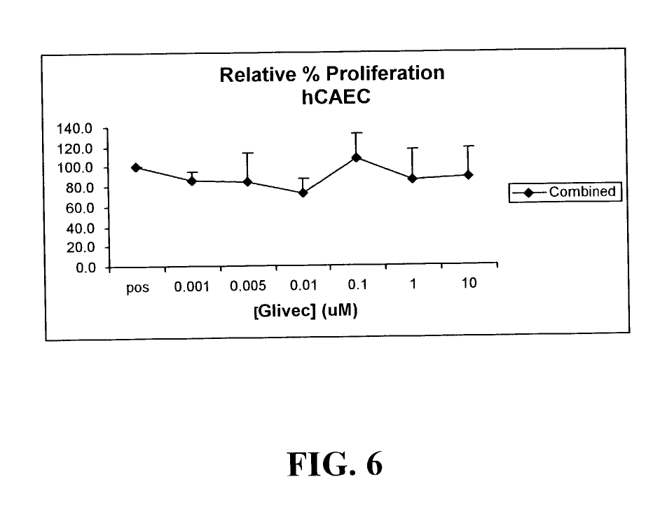

The results are shown in FIG. 6. As can be seen from the figure, STI-571 did not have a significant effect on the proliferation of human coronary artery endothelial cells at any of the STI-571 concentrations tested. This Example, in conjunction with Examples 3-5, are significant because they show that STI-571 has a profound inhibitory effect on human vascular smooth muscle cells (inhibits proliferation, DNA replication, and cell migration), but does not inhibit the proliferation of endothelial cells. This is notable because the proliferation of endothelial cells around an inserted vascular stent is desirable so that the stent becomes firmly implanted within the vessel wall.

What is claimed is:

1. A device for stenting a blood vessel, the device comprising:
a cardiovascular stent, autologous venous/arterial graft, prosthetic venous/arterial graft, vascular catheter, or vascular shunt having coated thereon, adsorbed thereto, impregnated therein, or covalently or ionically bonded thereto an amount of a protein tyrosine kinase inhibitor; the amount being sufficient to prevent or inhibit proliferation of vascular smooth muscle cells in an area within a blood vessel immediately adjacent to and/or proximal to the cardiovascular stent, autologous venous/arterial graft, prosthetic venous/arterial graft, vascular catheter or vascular shunt, while simultaneously not inhibiting the proliferation of vascular intimal cells.

2. The device of claim 1, comprising a cardiovascular stent,

3. The device of claim 1, comprising an autologous venous/arterial graft.

4. The device of claim 1, comprising a prosthetic venous/arterial graft.

5. The device of claim 1, comprising a vascular catheter.

6. The device of claim 1, comprising a vascular shunt.

7. The device of claim 1, wherein the protein tyrosine kinase inhibitor is a platelet-derived growth factor inhibitor.

8. The device of claim 7, wherein the protein tyrosine kinase inhibitor is also a Bcr-Abl tyrosine kinase inhibitor.

9. The device of claim 1, wherein the protein tyrosine kinase inhibitor is STI-571.

10. The device of claim 1, wherein the protein tyrosine kinase inhibitor is 4-{(4-methyl-1-piperazinyl)methyl}-N-{4-methyl-3-{{4-(3-pyrimidinyl}amino}-phenyl}benzamide and/or a pharmaceutically-suitable salt thereof; the amount being sufficient to prevent or inhibit proliferation of vascular smooth muscle cells in an area within a blood vessel immediately adjacent to and/or proximal to the cardiovascular stent, autologous venous/arterial graft, prosthetic venous/arterial graft, vascular catheter or vascular shunt.

11. A device for stenting a blood vessel, the device comprising:
a cardiovascular stent, autologous venous/arterial graft, prosthetic venous/arterial graft, vascular catheter, or vascular shunt having coated thereon, adsorbed thereto, impregnated therein, or covalently or ionically bonded thereto an amount of 4-{(4-methyl-1-piperazinyl)methyl}-N-{4-methyl-3-{{4-(3-pyrimidinyl}amino}-phenyl}benzamide and/or a pharmaceutically-suitable salt thereof; the amount being sufficient to prevent or inhibit proliferation of vascular smooth muscle cells in an area within a blood vessel immediately adjacent to and/or proximal to the cardiovascular stent, autologous venous/arterial graft, prosthetic venous/arterial graft, vascular catheter or vascular shunt, while simultaneously not inhibiting the proliferation of vascular intimal cells.

12. The device of claim 11, comprising a cardiovascular stent,

13. The device of claim 11, comprising an autologous venous/arterial graft.

14. The device of claim 11, comprising a prosthetic venous/arterial graft.

15. The device of claim 11, comprising a vascular catheter.

16. The device of claim 11, comprising a vascular shunt.

17. A method of preventing restenosis following vascular intervention, the method comprising coating, adsorbing, impregnating, or covalently or ionically bonding to a cardiovascular stent, autologous venous/arterial graft, prosthetic venous/arterial graft, vascular catheter or vascular shunt used in the vascular intervention an amount of a protein tyrosine kinase inhibitor; the amount being sufficient to prevent or inhibit proliferation of vascular smooth muscle cells in an area within a blood vessel immediately adjacent to and/or proximal to the cardiovascular stent, autologous venous/arterial graft, prosthetic venous/arterial graft, vascular catheter or vascular shunt, while simultaneously not inhibiting the proliferation of vascular intimal cells.

18. The method of claim 17, wherein the cardiovascular stent, autologous venous/arterial graft, prosthetic venous/arterial graft, vascular catheter or vascular shunt is coated with a platelet-derived growth factor inhibitor.

19. The method of claim 17, wherein the cardiovascular stent, autologous venous/arterial graft, prosthetic venous/arterial graft, vascular catheter or vascular shunt is coated with a Bcr-Abl tyrosine kinase inhibitor.

20. The device of claim 17, wherein the cardiovascular stent, autologous venous/arterial graft, prosthetic venous/arterial graft, vascular catheter or vascular shunt is coated with STI-571.

21. The device of claim 17, wherein the cardiovascular stent, autologous venous/arterial graft, prosthetic venous/arterial graft, vascular catheter or vascular shunt is coated with 4-{(4-methyl-1-piperazinyl)methyl}-N-{4-methyl-3-{{4-(3-pyrimidinyl}amino}-phenyl}benzamide and/or a pharmaceutically-suitable salt thereof.

22. A method of preventing restenosis following vascular intervention, the method comprising coating, adsorbing, impregnating, or covalently or ionically bonding to a cardiovascular stent, autologous venous/arterial graft, prosthetic venous/arterial graft, vascular catheter or vascular shunt used in the vascular intervention an amount of 4-{(4-methyl-1-piperazinyl)methyl}-N-{4-methyl-3-{{4-(3-pyrimidinyl}amino}-phenyl}benzamide and/or a pharmaceutically-suitable salt thereof, the amount being sufficient to prevent or inhibit proliferation of vascular smooth muscle cells in an area within a blood vessel immediately adjacent to and/or proximal to the cardiovascular stent, autologous venous/arterial graft, prosthetic venous/arterial graft, vascular catheter or vascular shunt, while simultaneously not inhibiting the proliferation of vascular intimal cells.


SEQUENCE LISTING (25 sequences)

SEQ ID NO: 1 - DNA, 3000 bp, Homo sapiens
atggggccggccccgctgccgctgctgctgggcctcttcctccccgcgctctggcgtagagctatcactgaggcaagggaagaagccaagccttacccgctattcccgggaccttttccagggagcctgcaaactgaccacacaccgctgttatcccttcctcacgccagtgggtaccagcctgccttgatgttttcaccaacccagcctggaagaccacatacaggaaacgtagccattccccaggtgacctctgtcgaatcaaagcccctaccgcctcttgccttcaaacacacagttggacacataatactttctgaacataaaggtgtcaaatttaattgctcaatcaatgtacctaatatataccaggacaccacaatttcttggtggaaagatgggaaggaattgcttgggggacatcatcgaattacacagttttatccagatgatgaagttacagcaataatcgcttccttcagcataaccagtgtgcagcgttcagacaatgggtcgtatatctgtaagatgaaaataaacaatgaagagatcgtgtctgatcccatctacatcgaagtacaaggacttcctcactttactaagcagcctgagagcatgaatgtcaccagaaacacagccttcaacctcacctgtcaggctgtgggcccgcctgagcccgtcaacattttctgggttcaaaacagtagccgtgttaacgaacagcctgaaaaatcccccggcgtgctaactgttccaggcctgacggagatggcggtcttcagttgtgaggcccacaatgacaaagggctgaccgtgtcccagggagtgcagatcaacatcaaagcaattccctccccaccaactgaagtcagcatccgtaacagcactgcacacagcattctgatctcctgggttcctggttttgatggatactccccgttcaggaattgcagcattcaggtcaaggaagctgatccgctgggtaatggctcagtcatgatttttaacacctctgccttaccacatctgtaccaaatcaagcagctgcaagccctggctaattacagcattggtgtttcctgcatgaatgaaataggctggtctgcagtgagcccttggattctagcaagcacgactgaaggagccccatcagtagcacctttaaatgtcactgtgtttctgaatgaatctagtgataatgtggacatcagatggatgaagcctccgactaagcagcaggatggagaactggtgggctaccggatatcccacgtgtggcagagtgcagggatttccaaagagctcttggaggaagttggccagaatggcagccgagctcggatctctgttcaagtccacaatgctacgtgcacagtgaggattgcagccgtcaccagagggggagttgggcccttcagtgatccagtgaaaatatttatccctgcacacggttgggtagattatgccccctcttcaactccggcgcctggcaacgcagatcctgtgctcatcatctttggctgcttttgtggatttattttgattgggttgattttatacatctccttggccatcagaaaaagagtccaggagacaaagtttgggaatgcattcacagaggaggattctgaattagtggtgaattatatagcaaagaaatccttctgtcggcgagccattgaacttaccttacatagcttgggagtcagtgaggaactacaaaataaactagaagatgttgtgattgacaggaatcttctaattcttggaaaaattctgggtgaaggagagtttgggtctgtaatggaaggaaatcttaagcaggaagatgggacctctctgaaagtggcagtgaagaccatgaagttggacaactcttcacatcgggagatcgaggagtttctcagtgaggcagcgtgcatgaaagacttcagccacccaaatgtcattcgacttctaggtgtgtgtatagaaatgagctctcaaggcatcccaaagcccatggtaattttacccttcatgaaatacggggacctgcatacttacttactttattcccgattggagacaggaccaaagcatattcctctgcagacactattgaagttcatggtggatattgccctgggaatggagtatctgagcaacaggaattttcttcatcgagatttagctgctcgaaactgcatgttgcgagatgacatgactgtctgtgttgcggacttcggcctctctaagaagatttacagtggcgattattaccgccaaggccgcattgctaagatgcctgttaaatggatcgccatagaaagtcttgcagaccgagtctacacaagtaaaagtgatgtgtgggcatttggcgtgaccatgtgggaaatacgtacgcggggaatgactccctatcctggggtccagaaccatgagatgtatgactatcttctccatggccacaggttgaagcagcccgaagactgcctggatgaactgtatgaaataatgtactcttgctggagaaccgatcccttagaccgccccaccttttcagtattgaggctgcagctagaaaaactcttagaaagtttgcctgacgttcggaaccaagcagacgttatttacgtcaatacacagttgctggagagctctgagggcctggcccagggccccacccttgctccactggacttgaacatcgaccctgactctataattgcctcctgcactccccgcgctgccatcagtgtggtcacagcagaagttcatgacagcaaacctcatgaaggacggtacatcctgaatgggggcagtgaggaatgggaagatctgacttctgccccctctgctgcagtcacagctgaaaagaacagtgttttaccgggggagagacttgttaggaatggggtctcctggtcccattcgagcatgctgcccttgggaagctcattgcccgatgaacttttgtttgctgacgactcctcagaaggctcagaagtcctgatgtga

SEQ ID NO: 2 - DNA, 1353 bp, Homo sapiens
atgtcagcaatacaggccgcctggccatccggtacagaatgtattgccaagtacaacttccacggcactgccgagcaggacctgcccttctgcaaaggagacgtgctcaccattgtggccgtcaccaaggaccccaactggtacaaagccaaaaacaaggtgggccgtgagggcatcatcccagccaactacgtccagaagcgggagggcgtgaaggcgggtaccaaactcagcctcatgccttggttccacggcaagatcacacgggagcaggctgagcggcttctgtacccgccggagacaggcctgttcctggtgcgggagagcaccaactaccccggagactacacgctgtgcgtgagctgcgacggcaaggtggagcactaccgcatcatgtaccatgccagcaagctcagcatcgacgaggaggtgtactttgagaacctcatgcagctggtggagcactacacctcagacgcagatggactctgtacgcgcctcattaaaccaaaggtcatggagggcacagtggcggcccaggatgagttctaccgcagcggctgggccctgaacatgaaggagctgaagctgctgcagaccatcgggaagggggagttcggagacgtgatgctgggcgattaccgagggaacaaagtcgccgtcaagtgcattaagaacgacgccactgcccaggccttcctggctgaagcctcagtcatgacgcaactgcggcatagcaacctggtgcagctcctgggcgtgatcgtggaggagaagggcgggctctacatcgtcactgagtacatggccaaggggagccttgtggactacctgcggtctaggggtcggtcagtgctgggcggagactgtctcctcaagttctcgctagatgtctgcgaggccatggaatacctggagggcaacaatttcgtgcatcgagacctggctgcccgcaatgtgctggtgtctgaggacaacgtggccaaggtcagcgactttggtctcaccaaggaggcgtccagcacccaggacacgggcaagctgccagtcaagtggacagcccctgaggccctgagagagaagaaattctccactaagtctgacgtgtggagtttcggaatccttctctgggaaatctactcctttgggcgagtgccttatccaagaattcccctgaaggacgtcgtccctcgggtggagaagggctacaagatggatgcccccgacggctgcccgcccgcagtctatgaagtcatgaagaactgctggcacctggacgccgccatgcggccctccttcctacagctccgagagcagcttgagcacatcaaaacccacgagctgcacctgtga

SEQ ID NO: 3 - DNA, 3768 bp, Homo sapiens
atggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgtccatcgtctctgcggtggttggcattctgctggtcgtggtcttgggggtggtctttgggatcctcatcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaagggccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtga

SEQ ID NO: 4 - DNA, 3429 bp, Homo sapiens
atggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaagacaagaaggatgaggagaaaaacaagatccgggaagagtggaacaatttttcattcttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagtgatgtccttaccacaccatggaaattcaaagttgccaaacagctggccagtgccctgagctacttggaggataaagacctggtccatggaaatgtgtgtactaaaaacctcctcctggcccgtgagggaatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttccagaaaaaaaaaccagccaactgaagtggaccccacacattttgagaagcgcttcctaaagaggatccgtgacttgggagagggccactttgggaaggttgagctctgcaggtatgaccccgaagacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcagcttatgagaaaatgctgggaattccaaccatccaatcggacaagctttcagaaccttattgaaggatttgaagcacttttaaaataa

SEQ ID NO: 5 - DNA, 3399 bp, Homo sapiens
atgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctagggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatga

SEQ ID NO: 6 - DNA, 1584 bp, Homo sapiens
atggcggggcgaggctctctggtttcctggcgggcatttcacggctgtgattctgctgaggaacttccccgggtgagcccccgcttcctccgagcctggcacccccctcccgtctcagccaggatgccaacgaggcgctgggccccgggcacccagtgtatcaccaaatgcgagcacacccgccccaagccaggggagctggccttccgcaagggcgacgtggtcaccatcctggaggcctgcgagaacaagagctggtaccgcgtcaagcaccacaccagtggacaggaggggctgctggcagctggggcgctgcgggacggggaggccctctccgcagaccccaagctcagcctcatgccgtggttccacgggaagatctcgggccaggaggctgtccagcagctgcagcctcccgaggatgggctgttcctggtgcgggagtccgcgcgccaccccggcgactacgtcctgtgcgtgagctttggccgcgacgtcatccactaccgcgtgctgcaccgcgacggccacctcacaatcgatgaggccgtgttcttctgcaacctcatggacatggtggagcattacagcaaggacaagggcgctatctgcaccaagctggtgagaccaaagcggaaacacgggaccaagtcggccgaggaggagctggccagggcgggctggttactgaacctgcagcatttgacattgggagcacagatcggagagggagagtttggagctgtcctgcagggtgagtacctggggcaaaaggtggccgtgaagaatatcaagtgtgatgtgacagcccaggccttcctggacgagacggccgtcatgacgaagatgcaacacgagaacctggtgcgtctcctgggcgtgatcctgcaccaggggctgtacattgtcatggagcacgtgagcaagggcaacctggtgaactttctgcggacccggggtcgagccctcgtgaacaccgctcagctcctgcagttttctctgcacgtggccgagggcatggagtacctggagagcaagaagcttgtgcaccgcgacctggccgcccgcaacatcctggtctcagaggacctggtggccaaggtcagcgactttggcctggccaaagccgagcggaaggggctagactcaagccggctgcccgtcaagtggacggcgcccgaggctctcaaacacgggttcaccagcaagtcggatgtctggagttttggggtgctgctctgggaggtcttctcatatggacgggctccgtaccctaaaatgtcactgaaagaggtgtcggaggccgtggagaaggggtaccgcatggaaccccccgagggctgtccaggccccgtgcacgtcctcatgagcagctgctgggaggcagagccgcccgccggccacccttccgcaaactggccgagaagctggcccgggagctacgcagtgcaggtgccccagcctccgtctcagggcaggacgccgacggtccacctcgccccgaagccaggagccctgaccccacccggtggcccttggccccagaggaccgagagagtggagagtgcggcgtgggggcactga

SEQ ID NO: 7 - DNA, 2544 bp, Homo sapiens
atggagcccttgaagagcctcttcctcaagagccctctagggtcatggaatggcagtggcagcgggggtggtgggggcggtggaggaggccggcctgaggggtctccaaaggcagcgggttatgccaacccggtgtggacagccctgttcgactacgagcccagtgggcaggatgagctggccctgaggaagggtgaccgtgtggaggtgctgtcccgggacgcagccatctcaggagacgagggctggtgggcgggccaggtgggtggccaggtgggcatcttcccgtccaactatgtgtctcggggtggcggcccgcccccctgcgaggtggccagcttccaggagctgcggctggaggaggtgatcggcattggaggctttggcaaggtgtacaggggcagctggcgaggtgagctggtggctgtgaaggcagctcgccaggaccccgatgaggacatcagtgtgacagccgagagcgttcgccaggaggcccggctcttcgccatgctggcacaccccaacatcattgccctcaaggctgtgtgcctggaggagcccaacctgtgcctggtgatggagtatgcagccggtgggcccctcagccgagctctggccgggcggcgcgtgcctccccatgtgctggtcaactgggctgtgcagattgcccgtgggatgcactacctgcactgcgaggccctggtgcccgtcatccaccgtgatctcaagtccaacaacattttgctgctgcagcccattgagagtgacgacatggagcacaagaccctgaagatcaccgactttggcctggcccgagagtggcacaaaaccacacaaatgagtgccgcgggcacctacgcctggatggctcctgaggttatcaaggcctccaccttctctaagggcagtgacgtctggagttttggggtgctgctgtgggaactgctgaccggggaggtgccataccgtggcattgactgccttgctgtggcctatggcgtagctgttaacaagctcacactgcccatcccatccacctgccccgagcccttcgcacagcttatggccgactgctgggcgcaggacccccaccgcaggcccgacttcgcctccatcctgcagcagttggaggcgctggaggcacaggtcctacgggaaatgccgcgggactccttccattccatgcaggaaggctggaagcgcgagatccagggtctcttcgacgagctgcgagccaaggaaaaggaactactgagccgcgaggaggagctgacgcgagcggcgcgcgagcagcggtcacaggcggagcagctgcggcggcgcgagcacctgctggcccagtgggagctagaggtgttcgagcgcgagctgacgctgctgctgcagcaggtggaccgcgagcgaccgcacgtgcgccgccgccgcgggacattcaagcgcagcaagctccgggcgcgcgacggcggcgagcgtatcagcatgccactcgacttcaagcaccgcatcaccgtgcaggcctcacccggccttgaccggaggagaaacgtcttcgaggtcgggcctggggattcgcccacctttccccggttccgagccatccagttggagcctgcagagccaggccaggcatggggccgccagtccccccgacgtctggaggactcaagcaatggagagcggcgagcatgctgggcttggggtcccagttcccccaagcctggggaagcccagaatgggaggagaaggtcccgcatggacgaagccacatggtacctggattcagatgactcatcccccttaggatctccttccacacccccagcactcaatggtaaccccccgcggcctagcctggagcccgaggagcccaagaggcctgtccccgcagagcgcggtagcagctctgggacgcccaagctgatccagcgggcgctgctgcgcggcaccgccctgctcgcctcgctgggccttggccgcgacctgcagccgccgggaggcccaggacgcgagcgcggggagtccccgacaacaccccccacgccaacgcccgcgccctgcccgaccgagccgcccccttccccgctcatctgcttctcgctcaagacgcccgactccccgcccactcctgcacccctgttgctggacctgggtatccctgtgggccagcggtcagccaagagcccccgacgtgaggaggagccccgcggaggcactgtctcacccccaccggggacatcacgctctgctcctggcaccccaggcaccccacgttcaccacccctgggcctcatcagccgacctcggccctcgccccttcgcagccgcattgatccctggagctttgtgtcagctgggccacggccttctcccctgccatcaccacagcctgcaccccgccgagcaccctggaccttgttcccggactcagaccccttctgggactccccacctgccaaccccttccaggggggcccccaggactgcagggcacagaccaaagacatgggtgcccaggccccgtgggtgccggaagcggggccttga

SEQ ID NO: 8 - DNA, 2640 bp, Homo sapiens
atgagtgattactgggttgttggaaagaagtctaactatgaagtattagaaaaagatgttggtttaaagcgattttttcctaagagtttactggattctgtcaaggccaaaacactaagaaaactgatccaacaaacatttagacaatttgccaaccttaatagagaagaaagtattctgaaattctttgagatcctgtctccagtctacagatttgataaggaatgcttcaagtgtgctcttggttcaagctggattatttcagtggaactggcaatcggcccagaagaaggaatcagttacctaacggacaagggctgcaatcccacacatcttgctgacttcactcaagtgcaaaccattcagtattcaaacagtgaagacaaggacagaaaaggaatgctacaactaaaaatagcaggtgcacccgagcctctgacagtgacggcaccatccctaaccattgcggagaatatggctgacctaatagatgggtactgccggctggtgaatggaacctcgcagtcatttatcatcagacctcagaaagaaggtgaacgggctttgccatcaataccaaagttggccaacagcgaaaagcaaggcatgcggacacacgccgtctctgtgtcagaaacagatgattatgctgagattatagatgaagaagatacttacaccatgccctcaaccagggattatgagattcaaagagaaagaatagaacttggacgatgtattggagaaggccaatttggagatgtacatcaaggcatttatatgagtccagagaatccagctttggcggttgcaattaaaacatgtaaaaactgtacttcggacagcgtgagagagaaatttcttcaagaagcctgccattacacatctttgcactggaattggtgcagatatataagtgatcctaatgttgatgcctgcccagaccccaggaatgcagagttaacaatgcgtcagtttgaccatcctcatattgtgaagctgattggagtcatcacagagaatcctgtctggataatcatggagctgtgcacacttggagagctgaggtcatttttgcaagtaaggaaatacagtttggatctagcatctttgatcctgtatgcctatcagcttagtacagctcttgcatatctagagagcaaaagatttgtacacagggacattgctgctcggaatgttctggtgtcctcaaatgattgtgtaaaattaggagactttggattatcccgatatatggaagatagtacttactacaaagcttccaaaggaaaattgcctattaaatggatggctccagagtcaatcaattttcgacgttttacctcagctagtgacgtatggatgtttggtgtgtgtatgtgggagatactgatgcatggtgtgaagccttttcaaggagtgaagaacaatgatgtaatcggtcgaattgaaaatggggaaagattaccaatgcctccaaattgtcctcctaccctctacagccttatgacgaaatgctgggcctatgaccccagcaggcggcccaggtttactgaacttaaagctcagctcagcacaatcctggaggaagagaaggctcagcaagaagagcgcatgaggatggagtccagaagacaggccacagtgtcctgggactccggagggtctgatgaagcaccgcccaagcccagcagaccgggttatcccagtccgaggtccagcgaaggattttatcccagcccacagcacatggtacaaaccaatcattaccaggtttctggctaccctggttcacatggaatcacagccatggctggcagcatctatccaggtcaggcatctcttttggaccaaacagattcatggaatcatagatctcaggagatagcaatgtggcagcccaatgtggaggactctacagtattggacctgcgagggattgggcaagtgttgccaacccatctgatggaagagcgtctaatccgacagcaacaggaaatggaagaagatcagcgctggctggaaaaagaggaaagatttctgattggaaaccaacatatatatcagcctgtgggtaaaccagatcctgcagctccaccaaagaaaccgcctcgccctggagctcccggtcatctgggaagccttgccagcctcagcagccctgctgacagctacaacgagggtgtcaagcttcagccccaggaaatcagcccccctcctactgccaacctggaccggtcgaatgataaggtgtacgagaatgtgacgggcctggtgaaagctgtcatcgagatgtccagtaaaatccagccagccccaccagaggagtatgtccctatggtgaaggaagtcggcttggccctgaggacattattggccactgtggatgagaccattcccctcctaccagccagcacccaccgagagattgagatggcacagaagctattgaactctgacctgggtgagctcatcaacaagatgaaactggcccagcagtatgtcatgaccagcctccagcaagagtacaaaaagcaaatgctgactgccgctcacgccctggctgtggatgccaaaaacttactcgatgtcattgaccaagcaagactgaaaatgcttgggcagacgagaccacactga

SEQ ID NO: 9 - DNA, 3213 bp, Homo sapiens
atgggagctgcgcggggatccccggccagaccccgccggttgcctctgctcagcgtcctgctgctgccgctgctgggcggtacccagacagccattgtcttcatcaagcagccgtcctcccaggatgcactgcaggggcgccgggcgctgcttcgctgtgaggttgaggctccgggcccggtacatgtgtactggctgctcgatggggcccctgtccaggacacggagcggcgtttcgcccagggcagcagcctgagctttgcagctgtggaccggctgcaggactctggcaccttccagtgtgtggctcgggatgatgtcactggagaagaagcccgcagtgccaacgcctccttcaacatcaaatggattgaggcaggtcctgtggtcctgaagcatccagcctcggaagctgagatccagccacagacccaggtcacacttcgttgccacattgatgggcaccctcggcccacctaccaatggttccgagatgggacccccctttctgatggtcagagcaaccacacagtcagcagcaaggagcggaacctgacgctccggccagctggtcctgagcatagtgggctgtattcctgctgcgcccacagtgcttttggccaggcttgcagcagccagaacttcaccttgagcattgctgatgaaagctttgccagggtggtgctggcaccccaggacgtggtagtagcgaggtatgaggaggccatgttccattgccagttctcagcccagccacccccgagcctgcagtggctctttgaggatgagactcccatcactaaccgcagtcgccccccacacctccgcagagccacagtgtttgccaacgggtctctgctgctgacccaggtccggccacgcaatgcagggatctaccgctgcattggccaggggcagaggggcccacccatcatcctggaagccacacttcacctagcagagattgaagacatgccgctatttgagccacgggtgtttacagctggcagcgaggagcgtgtgacctgccttccccccaagggtctgccagagcccagcgtgtggtgggagcacgcgggagtccggctgcccacccatggcagggtctaccagaagggccacgagctggtgttggccaatattgctgaaagtgatgctggtgtctacacctgccacgcggccaacctggctggtcagcggagacaggatgtcaacatcactgtggccactgtgccctcctggctgaagaagccccaagacagccagctggaggagggcaaacccggctacttggattgcctgacccaggccacaccaaaacctacagttgtctggtacagaaaccagatgctcatctcagaggactcacggttcgaggtcttcaagaatgggaccttgcgcatcaacagcgtggaggtgtatgatgggacatggtaccgttgtatgagcagcaccccagccggcagcatcgaggcgcaagcccgtgtccaagtgctggaaaagctcaagttcacaccaccaccccagccacagcagtgcatggagtttgacaaggaggccacggtgccctgttcagccacaggccgagagaagcccactattaagtgggaacgggcagatgggagcagcctcccagagtgggtgacagacaacgctgggaccctgcattttgcccgggtgactcgagatgacgctggcaactacacttgcattgcctccaacgggccgcagggccagattcgtgcccatgtccagctcactgtggcagtttttatcaccttcaaagtggaaccagagcgtacgactgtgtaccagggccacacagccctactgcagtgcgaggcccagggggaccccaagccgctgattcagtggaaaggcaaggaccgcatcctggaccccaccaagctgggacccaggatgcacatcttccagaatggctccctggtgatccatgacgtggcccctgaggactcaggccgctacacctgcattgcaggcaacagctgcaacatcaagcacacggaggcccccctctatgtcgtggacaagcctgtgccggaggagtcggagggccctggcagccctcccccctacaagatgatccagaccattgggttgtcggtgggtgccgctgtggcctacatcattgccgtgctgggcctcatgttctactgcaagaagcgctgcaaagccaagcggctgcagaagcagcccgagggcgaggagccagagatggaatgcctcaacggtgggcctttgcagaacgggcagccctcagcagagatccaagaagaagtggccttgaccagcttgggctccggccccgcggccaccaacaaacgccacagcacaagtgataagatgcacttcccacggtctagcctgcagcccatcaccacgctggggaagagtgagtttggggaggtgttcctggcaaaggctcagggcttggaggagggagtggcagagaccctggtacttgtgaagagcctgcagagcaaggatgagcagcagcagctggacttccggagggagttggagatgtttgggaagctgaaccacgccaacgtggtgcggctcctggggctgtgccgggaggctgagccccactacatggtgctggaatatgtggatctgggagacctcaagcagttcctgaggatttccaagagcaaggatgaaaaattgaagtcacagcccctcagcaccaagcagaaggtggccctatgcacccaggtagccctgggcatggagcacctgtccaacaaccgctttgtgcataaggacttggctgcgcgtaactgcctggtcagtgcccagagacaagtgaaggtgtctgccctgggcctcagcaaggatgtgtacaacagtgagtactaccacttccgccaggcctgggtgccgctgcgctggatgtcccccgaggccatcctggagggtgacttctctaccaagtctgatgtctgggccttcggtgtgctgatgtgggaagtgtttacacatggagagatgccccatggtgggcaggcagatgatgaagtactggcagatttgcaggctgggaaggctagacttcctcagcccgagggctgcccttccaaactctatcggctgatgcagcgctgctgggccctcagccccaaggaccggccctccttcagtgagattgccagcgccctgggagacagcaccgtggacagcaagccgtga

SEQ ID NO: 10 - DNA, 3645 bp, Caenorhabditis elegans
atgggtcattcacatagtactgggaaagaaatcaatgacaatgaactcttcacatgtgaagatcctgtattcgatcaaccggtggcgagtccgaaatcggagatttcgagcaagttagccgaagaaatagaacggagcaaaagtccactcatactcgagatgttccgtccaacatttgacacatttcgaccgccgaacagtgacagctcgactttccgtggcagccagagcagagaggatctagtagcatgtagctcaatgaattcggtaaacaacgtgcacgatatgaatacagtttcctcttcatcatcatcatctgcaccactttttgtagctctctatgatttccacggtgtcggcgaagagcagctttcgttacgaaagggtgatcaggtgcgaattctgggttacaacaaaaacaatgagtggtgtgaggcacgattatactcaacgagaaaaaatgatgcgagcaatcagcgaaggttaggcgaaattggatgggtgccaagtaattttattgctccgtacaactctttggataagtacacgtggtatcatggcaaaatctcaaggagcgattctgaggctatactaggcagtggaatcactggctcatttttggtacgagaaagtgaaacaagtataggacagtatacaatctctgttcgccatgatggtcgagtgtttcactaccggatcaatgtagataatacagaaaagatgttcatcacacaagaagtcaaattccgcacacttggagagttagtgcaccatcatagtgttcacgctgatgggctgatatgtcttttaatgtacccagcgagtaaaaaggacaagggacgtggactgttctcactgtcgcctaacgcgccagacgaatgggaactagatagatccgaaatcatcatgcataacaaattgggcggtggacagtacggagacgtgtacgagggatactggaaacgacatgactgcacaattgcagtgaaagcgttgaaggaagatgcaatgccacttcatgaatttttagcagaagctgctatcatgaaagatttgcaccacaaaaaccttgttcgactgcttggagtatgcactcacgaggcaccgttctatattatcaccgagtttatgtgcaatggaaatttgctcgagtacctgaggaggaccgataaaagcttgctgccacctataatccttgttcaaatggctagtcagattgcgtccggcatgtcgtacctggaagccagacacttcattcatagggatttggccgcaaggaattgcttagtatccgagcataatattgtaaaaattgccgactttgggttggcaagattcatgaaggaagacacctatacagcacatgctggagccaagtttcctatcaaatggactgccccagaggggcttgcattcaacaccttcagctctaaatctgatgtttgggcgtttggagttctgctctgggaaattgccacgtatggaatggctccctatccaggcgtcgagctgtcaaatgtttatgggcttttggaaaacgggttccgtatggatggcccgcaagggtgccctccatcggtgtatcgccttatgcttcagtgctggaactggtctccgtcggatcgtcctcgtttccgagatattcatttcaacttggaaaatctaatttcaagcaattccttgaacgacgaggtgcaaaaacaattgaaaaagaataatgataagaaactggaaagtgacaaaagaaggtctaacgttagagaacgaagtgactctaaatccagacattcttcacatcacgaccgtgaccgtgaccgggaatctcttcattctcggaactcaaatcctgaaattcccaatagaagttttataagaaccgacgacagtgtatcattcttcaatccatcaaccacaagtaaagtaacgtcgtttcgtgctcaaggaccaccgttcccaccaccgccacaacaaaacacaaaaccgaaactattgaagtcagttctgaatagtaacgctcgtcatgcatcagaggagtttgagagaaacgaacaagatgacgtggttcctttggccgagaaaaatgtgcggaaagcggttaccaggctgggtggaactatgccgaaaggacaaaggatagatgcatatttagactcgatgagaagggttgacagttggaaagaaagcactgacgctgacaatgaaggggcgggatcatcatcgctgagcagaactgtatcgaatgattctcttgacacacttcctctgccagattctatgaactcgagtacgtatgttaaaatgcatcctgcatccggcgagaacgttttcctgagacaaattcgttcaaaactgaagaaacgaagtgagacaccagagttggatcatattgattcagatactgccgatgaaacaacaaaatcggaaaagtcaccctttggatctttgaataaatcttctatcaaatatccaattaaaaacgcgcccgaatttagtgagaatcactctagagtcagccctgtcccggtgccaccatctcgtaacgcttctgtaagtgtaagacccgattcgaaagcagaagactcatcggatgagacaacaaaagatgttggaatgtggggtcctaagcatgccgtgacgcggaaaattgaaattgtcaagaatgattcgtatccaaatgtagaaggcgagttgaaagcaaaaattcgaaatttacgtcatgtacccaaagaagagagcaacacaagtagtcaagaagatttgccacttgatgcgacagacaacacaaatgacagcatcattgtgattccaagagatgaaaaagcaaaagttcgtcaactggtgacacaaaaagtatctcctcttcaacatcatcggccattctcactgcaatgtccaaacaattctacaagctctgcaatatcgcattctgaacacgcggatagctcagaaacatcttcactttccggtgtctatgaggaacgtatgaaacctgaacttccaagaaaacggagtaatggcgatacaaaagtggtgccagtaacatggattatcaatggagaaaaggaacccaatggtatggctcgaacaaaatctctacgtgatattacatcaaagttcgaacagcttggaacagcttccacgattgaaagtaagattgaagaagccgtcccatatcgtgagcatgcattggaaaagaaaggaacttcaaaacgattttcaatgctggaaggaagtaatgagttgaagcatgttgtcccaccgcgtaaaaaccgaaaccaagacgaatctggctcaattgatgaagaaccagtgagcaaggacatgattgtatcgttgctcaaagtaatccaaaaggaatttgtgaatcttttcaatttggcgagctcagagatcactgatgaaaaactacaacaatttgtaataatggctgataatgtacaaaaacttcattccacgtgttccgtctatgcagaacaaatctcaccgcatagtaaatttcggttcaaagaacttctttctcaacttgaaatctacaatcgacaaattaaattttcccacaaccctcgagcgaagccagttgatgacaaacttaaaatggcgttccaggactgtttcgaccaaatcatgaggctggtggatcgctga

SEQ ID NO: 11 - DNA, 3672 bp, Caenorhabditis elegans
atggcaagcacgtcaggggcgcttgtcgacgacaacgtcctcgaagtgctccgcaaagcacagttggacgcatttattagtcagtttgtcttcttattcaacgtcagaaggtttgatcacttttcacatgttcgagataaagatatgctggaaattggtatgcaacaagttcaaattcggcagctccgagagcagattctcaaaatgtccagagaaatgtggaatcggagtgatccgaagcaagtgtacattcaagccgatcagtcgatgccagcacaaaattcgattgacgagaaagcactgattccaaatgagcagattaaactgtacgagttgattggcgagggctcttttgctgtggtgaagcgtggtacgtggacacagagcaatgggacgcatgtgaatgtcgctgtcaaaattctccgcgacatttctccaaatattatggatgatttgagagtggaagccagtcatttgctcaagctccagcacccgtctttgattcgcctttacggaattgttcgccagccagcgatgatggtgtttgaactctgtgaaggtggttcactgctcgacagactacgagatgacaaaaaggcaattcttctggtgtcacggcttcatgactattgtatgcaaattgcgaaggctttgcagtttttggagtcaaaacactgtgtacacagagatgtggcagcaaggaatattttgttggctagagacgaaaggacagtcaagatctgtgattttggactcatgcgagcactaaaagaaaatgagcaaatgtacactatggctccacaaaagaaagtcccatttgcctggtgccctccggaagcacttcgtcatcgcaagttctctcatgcttccgacgtctggtcgtacggagtcaccatctgggaggtgttcacatttggcgaggagccatgggtcggctgtcgagccatcgatgtgctcaaaaacattgacgccggcgagaggctggagaagcccaagtactgctcggagcgaatttatcaaatcatgaagaattgttggaaattcaatccggcagagcgatgcaaatttggtgcaattcgagaggacttggtggcggccatgtttttggatgcagtggcaagggagacgtacaactctattcaaccgggcgcactacaattgacaaaaggggatgaagttgttgtggtggagaacacaggccaagactggtttggtcagaacaagaagaaccaaaagtttggcacattcccccgatcagttgtgtttgcgcagacgaacaacgcggttgcggcagcgacggcggttaccccacagaaagttccaacggcgccaacgatcagaattccaccgtcacacccacccccagccccgctgaaaccattgaacaataatacgaaaacttcgctgaacgaccgcacgtcaaaaatttcaatgcctgtggcaggttctttcatccataccggtcacggagacccactaggaggccaatcatggggtaacccagctacgattgcggacatgtatctcaagaatccagtgaacggcgctccattgtctagtatgtcgagtggtgcggaaattatcgccagtaaggagttgctcaccaatggcggccggagcacacaccaacctgctgctccatcgcctgccgtcatgtccaagattcgaggtctttcgcttgatttgccagaatatgatgatttcgatcgagcattcgatgatgggttttctccgtcgaagatcgagctgcccagagagttttgtggcaatgacagcgtaatcagtggtgggtcgaacagcatcggcttggctaacacttatgtcatggaaccgcccaagcaggcatttgatattcgaggaaatcgagtgctcccgccaacgaacaaggcgcctgtgctcattccaactaacccggcgccaagtgtcatctcgagcacagcttctgcaggaatcacactttctacgaacagttctcagatgtttaccagtcaagaccgccattcgaatatgcccgcaaatcttttccccgagcttcaacaccgcctcaatcaaggaagttcaacgggaaatggcgtccgacctcggccagcttcctcgattggaattcaaaacaatgatttgagcatgctcaaccctcaacaaccagcgaatattccgtgcctggttccaactccggctccaccagctccagcacacttttctcaaccggtgtcttcccagagagttgcacaacaacaacagaacactttgcaaaaagcgctgaacgatgaactcaaaggaaatctgaacaaaagacctactggcacgacggcaccaccgtcaaatgggttcaatgctccacgagcagacgttgcaccggtccaacagcgaccgatctcatcggcatctattccagcgctccaaccacaacccattcaacacattcagaagcctatccaaccgcaacaagttcgtataccgccatcaacagctcccgttcagaaaccagttcaagtctcagctcctacccatagtaatgtggcacccacaacttcatctcaagcgtctgcagatgcacgcaatccgctacctccaaaaacaagcccaccagttagcaacacgcctatcacagttgctcctgttcacgcggcaccaactacttcggcaccatcaacttcggtggtaacgagaaggccaacttcaaccacagctcaaatgtcggacgaggagagacggtcaagaattgccatggacatcagctctgcacttccagctcccagtgctttgctctatggatctaactccacatcatcacttccgtcagcggcagtgtctacagcgtcttctgtgccatcaactgcaagagacaatccagtggaaacaagaccatctcaacctcatgttaccatgccacccaaaaaatcttctgagccgattctctcgtctgaggtgctccaaccaactcgtctgccatctgccacaacttcgcaggcaaaaccagtgactcaaccaatccgtcacccatcacctccggtagccactgttataccgactgcagtggttgacaaaaagccagtttcacaaaatcaaggaagcaacgttcctttgtttaacattaccaactccagcaacgggtaccctcagttaaatggatatccaaactatggaaacggttttcaggcgtatggttatggaatgaactatcatcaaggatatcctggatatcaaggatacaattcatatggcaacggaatggggcagcttgcactgacccacaacgccgtcacttctttgccaccgttggttccatcagagaacagattctccggaacagcccaaccacttggcgagtctgacattatggagtttttgggaacacagcaacgtcaagcgggttcttcatcgcgagcagttccacctgcatctgcatccacgtcagcagcttctggaatcacggatttgagtatggcagataagatggaggtgttgtatagagaagctgattttacgcataaaggaaattgtgataccatggtttctcagtgcaacggaaacaccgaacaggcgttgaagcttctcaaacaacaacacttggtggatatggaacttgcaatgtcaacggagaccgcccgacaagcactcgaggccagacagtatgatctccctgcagccgccaacatgttgctcggctga

SEQ ID NO: 12 - DNA, 1335 bp, Caenorhabditis elegans
atgtcaattaattctctttcgaacgaaacgcccactccaacaatcgagaaagaagcctacttccatggattgatccaacgagaagatgtcttccagctccttgacaataatggcgactacgtggtcagactgtcggatccaaagcccggcgagcctcgctcctacattctgagcgtcatgttcaacaataagctcgatgagaacagttcggtgaagcactttgttatcaattctgtagagaacaaatattttgtgaacaacaatatgtcgttcaacacgattcaacaaatgctcagccactatcagaagagtcgcacggagattctcgaagcgtgcaagattttgcatcctgtgcgcagacaattctgggagttagatcatggcaatatcgtgattgagaagaaactaggcgaaggtgcttttggtgaagtttcctccggagttatgaagttcaagagaggtggaaggctggtgaaggttgctgtgaagcaggtaaaaaccgatggtatcgggaaagatcaaatcaaggatttcctgatggaagctcgtaccatgcgaaacctcggtcatccaaacatcgtaagattcctcggaatcgccgtgctgcaggagccgctgttcctggtgatggagctcgcgacgggcggcgctttggatagctacttgaagcataatgagttgctgccgattgacaagagacacgagatgcttcttcaagcagcatggggtctcgagtacatccatggaaaacccatgctgcatagggacatcgccgcgcgaaattgcctttatggagatgggaaggttaaaatttcggatttcggcctaacccgtagaggaaccatctaccaattgcatccggagacgaagtcaccaattcgatggctggcagttgaaactatcaggactatggtttgctctcagaagactgacgtctgggcttacgggattctctgctgggagatcttcaacaacggagccgagccgtatccgggactgactgccaatgaggttgctaagcaggtgactgatggataccgtatgccaccacaccagttggctgcgccagaggttcaagcgttgatgacgagatgctgcgcggagaaccccaacgatcgtccaacaatgtcggatgtcgctcagatcttgcaacgcgtcactggtcaaggacgtcccaactttgcagcgattgccaagaaagaggctgaagagcttctcatcatgaattctcgtagtgcaaggagaacttcacgacgtaagggcagtaataagaagtcggcaattccaaacggagttttaacacctgtcaatagagctcaagaaattaagcattga

SEQ ID NO: 13 - DNA, 1689 bp, Caenorhabditis elegans
atgttcatcagcaaagaggaaatgaatcgtacttttggtgtcaaagctgagctgaattacattgaaatggggaatgttagctcgtactctacaaagtttcactacagagttatggcaaacatcgactacctctcgttcacatggaatgctgttggaattgtacactatgaagtttacgtcgaatctgatgactcttctgtgcttcctattgttcgaattccattgaaaggaacggtgccagaatctttgcaggacttcaccgttgaatacagatgtgccggacaccgatccggacaatttgctgtcagtctatatttcacattcaaatatggtaataaggagccgttgaaagtgaaattgcgacaggagaagatctgcgcttcaagggacggacgtcgaggtctgaacggaggctacgagggtcatgaagtcgacgacactgactcaatagacaaggcattttttgttatcatttgcattgctgcggcattcctacttattgtggcagcaacgttgatctgttatttcaagcgctctaaaaaagaagacatgattccgactcgacttccaacgtcttttcggaattctttgaaatctacaaaaagcgcgcagccttttcttctgagcacaccgcgagatggacctccgactctttccgctatttcaagcgctccttgttcttcgtcgtctgcgtcgggaaattcgataatcccgagcaagccaagaaacattgacgtgagacgtgcattgttacaactctatcaagatcgagatgcttttcaatctctacctctagatatggagggaacatttggagaagtgagatatgcaatttggcgtcaagtagatgacgtactgaacggagatgttgacgacgaagaagacacattctgtaaccaggaagctgtttacaccaaaacgttgaaaaataatgcctcaccaattcagctggatcggtttttgtccgacgcccttctattttacaacatcacacctcaccaaaacttgtctcaagtggcatgtgtggcttccttcggaagattcgaccgcccggaaactgtcacagattttccacttgtttgttacagacaccaaggctttggaaacctgaagaagttcctcaccatctgccgacatggtgataaaactaaaggagctcaaactctccgaactcatcaactcgtctctctggccacacaagtatcttctgcagtagctcatatacacaaatatagaatagtgcataacgacattgccgctagaaactgcttgatcgcagaagtgaatgggcgactccaagtgcaattatgcgactcggcgctgtcccgcgatctgttcccagctgattatcactgcttgggtgacaatgagaacagaccattgaaatggatgtctccagaagctattgcaaatgagctgtactcatcggccgctgatgtttggtcactgggagttctactgtgggagctcatgtcgctaggaggatctccacacgctgaaatagaccctgaggaagtgtacacaatgattctcaaaggaaagcgtctgcaacagccgaacaattgtccggatcaattatacgaagtcatgctgtgctgttggagggtactcagcgaagatcgtcctagcagtgagcaggtagttcatggacttcgagactttaacattcaactcagtcaatacatctaa

SEQ ID NO: 14 - DNA, 3603 bp, Drosophila melanogaster
atgaacaccgcgggagccaccagtcaaccgccgcccactaaaaatgagattaactccgaggagtatctcatccacgtgcatatgccgaacaagagcttcaaggctgttcggtttaatgtcaaggagaccgttttccatgtgatccggcgcactgtcgaggatctgggcacggatggacggacgcccagcattcagcgatatgcctgccgcatgcttaacatgatcaccaaggaggtgatttggctggctagaagcacttcaatgcagaaggttctctcgcacatcctgacgcccggctgctccaacgttgactgtcccaacaaccagtcggagttggatgaggttctattggagcacggaagaaggatcaccgataatagggtgtggcgagtggagctcagagtgcgctacgtgccaaataatattcaagagctcttcgaggaggacaaggccacatgcttctattatttcaatcaggtgaaagaggactttatccaagccaatgtcacagccatcgacactgaagtggcggtgcaactgtgctgtctgggcattcgtcattatttcaagaacatcaccgtgaaagcacctgacaaaaagcagcacattgactacattgaaaaggaaatcggatttaaaagttttcttccacaatctgtgatagccacatcaaagccaaagaatcttaagaaactgatccaagtcggttacaaaaaggtctacaattacaacgacattgagtacttgacgcggttctttgatcttctgaagaatatttatttaacgaacttcgagcagttctcggtaaccctgagctcggcgtggaatatttctggaattctacacgtcggccctcacattggaatctcgtaccagactcatcctcaggccagcttgaagaacgtggctcagtttaaagatgtggtctctattaaaacgtgcactttaccaaaggaaaaactgtccaagtctggggagaataccacagaaccagagcttcagaattttaattgcaactgccagaagattaaaacccaaataaaaatatccgcttccaacaatgtggaagatttggttataacgtgcaatggtattaataccgctgagagtattgctgacctaattgacggttactgccggctgttatcaaaagacctagagttcacgatttggcatcgagagacaaacgcgtcgaacgaagatagcgcaaaagcattgcccaatgatgcgacgctggggtccaataaatcaacttcaagtcagggaaaaccgatgctgaccgatgattatgccgagattggtttattggagggcgagggcgactactctacgcccaccgttcgaaattatgagttggacagagccctcataacgccgagcgccaaaattggtgtgggacagtttggtgatgtgtatgtaggcacgtatacgcttccgaaactgggcaagggcaagaacttagcaggaaatggaaaaaatagtaatagtgaccaaagaaatgccgattcaaggccagatgttatacaagtggcgataaagacatgtaaagctaacgacgatcctgaaaaaaccgaaaattttcttgccgaagcttatattatgcaaaaattcgatcatccccatattatacgcttaatcggcatttgcagcgtaatgcccatttggatagttatggaattggccaaactgggtgaattgcgtgcgtacttaaagacaaacagcgaaagattaagccacggtactttactgaagtattgctatcagctatcgactgctcttagttatttggaatccaaaaagtttgttcaccgagatatagcggcgcgtaatgtactagtcagctcaccaacgtgtgttaagttggctgattttggattatcacgttgggtttccgatcagtcgtattatcactcaacacccacagttgccctacccattaaatggatgtcccccgagtcaataaactttagaagatttaccactgctagtgatgtttggatgtttggtgtctgcatttgggaaatactcatgctcggtgtaaagcctttccaaggcgtcaagaacagcgatgttatattgaagctcgaaaacggagagcgtctgccattgcctcccaactgcccacctaggttatattcgttaatgtcccaatgctgggcgtacgagccacttaaacgaccgaatttcaagcggatcaaggaaactctgcatgaaattctgattgaagacagcattaattcatcggagacactgaagcgggagcaacgaaaagtggcttccatgtcctggattggcagtgatgacatcgacattccgccatcgaaaccttcaagggtgatgcacgatcctgacatcactggcttaatgcctgaaacaacggggctacctcagacctatattattgcacaaaatcccgcggtgctggccaaactgatgatggagaaccaaaaacgaggcataaatccagcggcgtacaccacaccagcttcgggcattcacaatgttttgggcgaaaaactacgacaacagcaaaaggatagcaacagcgacagcgaatggttaattcaagaagaattgctacggcagagatcctgctcaatacctcaaggatcgctcaatgatcatcaggctcaaatgtttaagcttgacttcatgtcagctggtccttccagtttgccggactgctcgaactccagttctcgacctatgacaccaaatgccaatctttcttcactgaagtcgaaccactcatcggcggatcatttgtccagcttgacatctgcagaagaacagatgggttcaaatgcacgaaacctgggcagtgcagttccaagtcgaccacctaaccgcgcagatgacgaagtttattgcgccaccacactggtggtcaaatcaataatggcgctgtcacaaggtgtggagaaagcgaataccgagggttacttggaattggttaagaacgtgggcgtcaagttgagaaacttgctaacatcggtggacaaaatatctataatatttccagcacaggccctcaaggaagtgcaaatggcacatcaggtactttcaaaagacatgcacgaattggtctcagcgatgcgattggctcaacaatatagtgacacaacgctggattgtgaatatcgcaagagtatgctgtctgctgcccacgttttggctatggacgccaaaaacctgtttgatgttgtcgattcgatacgtcaacgttatcagcatctattcccgccatccgccacaaaagaaacaagttgttcgtcaagtttcgagtcgacttctggatctattgtcgcagagccagttaatgaccttggtggttatatcaagactagcacttctggagatttgcttcaaaacacaggaatatatgataatgatttgcatcatagcttcaactcgcaattgcagttgcaaaacccaaaagccagcatcgacttaagcggcggtggtagtctacagcgagggatgagccttggcttggacacaaccaggtcgacaaacgaaccgttgcgaattgttgaggagaccctgggcagcccgggtgaacatatgtactgcaatacgtccgccttgcacggccacgcgtaa

SEQ ID NO: 15 - DNA, 2772 bp, Drosophila melanogaster
atgcttattttctacgcgaagtacgcatttatcttctggtttttcgtgggaagcaatcaaggtgaaatgttgctaatggacaaaatctctcacgataagacgcttctcaacgtcaccgcttgcacccagaattgtctggaaaagggccagatggatttccgaagctgtttaaaggactgcaggattaatggaacatttcccggggctctgcgcaaggtgcaggaaaactaccagatgaacatgatctgccgcacggagtcggaaatcgttttccaaatagattgggtgcagcacagcaggggaaccgagccggctccaaatgccacctacataatccgggtggatgctgtcaaggacgacaacaaagaaactgcgctttacctgtctgatgacaactttctcatcctgccgggattggagtccaactctacccacaacatcaccgccctggcgatgcacggagatggcagctactccttgatagcaaaggaccagaccttcgccaccctcatccgaggctatcagcccagcaaaatgggagcggtgaatctgctgcggtttgtcccccaaccagacgacctgcatcacattgctgccgaaatcgagtggaagccatcggcggagagcaactgctatttcgacatggtgtcgtattcaaccaacagcgtgaatatggacgagccactggaggtgcagttccgggatcgcaaaaagctgtacaggcacacggtggacaacttggagtttgacaaacagtatcacgttggcgtaagaacggtgaacataatgaatcgactggagagcgatctgcagtggctgccaatcgctgttccaagctgcttggattggtatccctataactacacactctgcccaccccataagccagagaatcttactgtgacccagaagcagtatctgccaaatattttggccctgaacatcacctgggcgcgtcccagatacctgccggataactatacacttcacatctttgatctattcaaaggaggtacggagctaaactatacacttgaccaaaacaggagccacttctatgtacccaagatcacggtactgggttcccatttcgaagtacatttggtggcccagtcggcaggcggaaaaaacgtatccggtttgacgttggacaaggttcatcgaggtgtgttgctgagcgagggcaacatggtcaagttggtactctttattatcgtgcccatatgctgcattttgatgctgtgctccctgacgttctgcagacgaaatcgttcggaggttcaggcgctgcaaatggacgctaaggacgcgaaggccagtgaatttcatctctccctgatggacagcagtggcctgctggtcaccctctcggccaacgagagtctggaagtaatggacgagctggaggtggagccacactcggtgctccttcaggatgtcctcggcgaaggagcctttggcttggtgcgacgtggagtttacaagaaacgccaagtggccgtcaagttgctgaaagatgaaccaaacgacgaggacgtatatgcgttcaagtgcgaaattcagatgctcaaggccgtgggcaagcatccaaatattgtgggtatcgtgggatactccactcgttttagcaaccagatgatgttgctaattgaatactgcagccttggaagcctgcagaactttttacgtgaggagtggaagttcaggcaggagcaaaatgcaattggacttaagaagaaccttgaacagaacgtggacaaccgacggtttaaccgactccctagaaattccatccatgatcgcatagaggatatcaacaactcgatgctgtccactgtggaagaggagagtgaatcggatcagacacactcaagtcgatgtgagacctacaccctcactcgaataaccaatgcagccgacaacaagggctatggcctggaggacattgaaaacatcggtgggagttacattcccaaaaccgctgaagctccaaaggatcggccaaaacggaagctgaagccgcagcccaagaaagactcgaagcaggatttcaaatcggacaacaagaagcgaatctttgagaacaaggaatactttgattgcctcgactcatcggataccaagccccgaataccactgaaatatgcagatttgctagacatcgcccaacaggtggcggtgggaatggaatttctggcccaaaacaaagtagtgcatagggatctggctgcccggaatgttctaatctccgtagatcgcagcatcaagatagcagattttgggctgagtcgagatgtgtatcatgagaacgtgtaccgaaagtccggaggaagtggcaagctgcccatcaagtggctcgcgctggagtccctcacccaccaggtgtacaccagtcagagcgatgtttggtcctttggtgtgctgctctatgagatcaccactctcggtggaatgccatatccgtcggtgtctcccagtgatctcttgcagctactgcgacaaggtcatcggatgaagcgaccggagggatgtacgcaagaaatgttttccctgatggaaagctgctggagctccgtgccatcacacaggccaacattttccgcccttaaacacagacttggtggcatgattttggccactaacgatgttccagaaaggctgaaacaactgcaagctgcaaccgagtcaaaattaaagtcatgtgacggtctaaacagtaaagtggagcaagtgccatgcgaggaagagctatacctagaacctttgaattaa

SEQ ID NO: 16 - DNA, 5229 bp, Drosophila melanogaster
atgttcaatatgccacggggagtgacaaaaagtaaatccaagcgtgggaaaattaagatggaaaacgatatggcagcagcagcaacaacaacagcctgcacgcttggacacatttgtgttttgtgccggcaagaaatgttgctggatacatgttgctgccggcaagcagtagaagcagttgacagccccgcaagcagtgaagaagcgtatagcagtagcaacagcagcagctgtcaagcaagcagtgaaatcagtgcggaggaggtctggtttctcagtcatgatgatatcgtactgtgccgcagaccaaaatttgacgaagtggagacgacgggtaaaaagagggacgttaaatgcagcgggcatcagtgcagcaatgaatgcgacgatggcagcacgaaaaacaatcgacaacagcgcgaaaacttcaatatctttagcaactgtcacaatattttgcgaacattgcaatcgctgctgctgctcatgttcaattgcggcattttcaacaagcgacgcaggcggcagcatcagcagcagcatcatcatcattatcagcatcatcatcagcagcatcatcagcagcatcatcagcggcagcaagccaatgttagttacacaaaattcctattgctgctacaaacactggcagcagcaaccacaagactgagtttaagccctaaaaactacaaacaacaacaacaactacagcataaccaacagctgccacgtgccacaccgcaacaaaagcaacaagagaaagataggcataagtgctttcactacaagcacaattactcttactcgcctggcattagccttctactctttatcctactggccaacacattggccatccaagcggtcgtgttgccagcacatcagcagcacctgctgcacaatgatatagccgatggactggataaaacagcgctttcggtgtcggggacgcaatcgcgatggacaaggagcgaatcaaacccaacaatgcgactgtcacaaaatgtaaaaccttgcaaatccatggacatcaggaacatggtgtcgcacttcaatcagctggagaactgcacggtcatcgagggcttcctgctgatcgatttgataaacgacgccagccctctgaacagaagctttccaaaactgaccgaggtcacagattatatcataatctaccgtgtgactggattgcactcgctgtcaaagatctttcccaatctgagcgtcattaggggaaacaagctgttcgacggatatgccttggtcgtctactcgaatttcgacctcatggatttgggacttcacaagctacgatccataaccagaggcggtgtgcggattgagaagaatcataagctgtgctatgataggaccatcgattggctggaaattctggcggaaaacgaaacccaactggtggtgctgacagagaacggcaaggagaaggagtgcaggctttccaagtgcccgggggagatcagaattgaggaggggcacgataccacggctattgagggagagcttaatgccagttgtcagctgcacaataataggcgcctgtgctggaacagcaaactctgccagacgaaatgccctgaaaagtgcagaaataactgcatcgatgagcacacctgctgcagccaggattgtttgggtggatgcgtgatcgataagaatgggaatgagagctgcatctcctgtcgaaatgtgtctttcaacaacatctgtatggactcctgtccgaaaggctattatcagttcgacagccgctgcgtaacggcgaacgagtgcatcacactgacaaagtttgaaacgaacagtgtgtattccggtattccatacaacggacaatgtatcacccactgtccaacggggtaccagaagtcagagaacaagcgcatgtgcgaaccttgtccgggcggcaagtgtgacaaggagtgctcctccggtcttatcgacagtttggagcgtgctcgggagttccacggctgcaccattataaccggaaccgagccccttaccatcagcattaaacgtgaaagcggcgctcacgtcatggatgaattaaaatatggcctggctgccgtccataaaattcagtcgtccctaatggttcatttgacctacggattgaagtccttgaaattctttcaatccctaactgaaattagcggcgatccgccgatggacgcggataaatatgctttgtatgtgcttgataatcgcgatctagatgagctctggggacccaaccaaacggtgttcattaggaagggcggcgtcttctttcatttcaacccaaaactatgtgtgtccaccattaaccagttgctgcccatgctggcctccaagccaaagttttttgaaaagtcagatgtgggcgcagactcgaatggaaaccgcggatcatgtggaacagccgttctcaatgtcacattacaatcagtgggagcaaactccgctatgctgaacgtcacgacaaaagttgaaataggagagccccaaaagccgagcaatgctacaattgtttttaaggatccgcgcgccttcatcggtttcgtgttttatcatatgatcgatccgtacgggaactcaactaaaagcagtgacgatccatgcgatgatcgctggaaggttagctctccggaaaagagcggggtcatggtattaagcaatttgattccgtacactaactactcctactacgttcggaccatggctatatcctcggaattgacaaacgcggagagcgacgtgaagaactttaggacgaatcccggacgaccgtcaaaggttacggaggtggtagcaaccgccatttcagattcgaaaattaacgtaacatggagctacctagataagccttatggcgtgctaacgcgctattttataaaagccaaacttataaatcggcctactcgaaacaataaccgggattactgtactgaacctctcgtcaaggccatggaaaatgacctgccagccacaacgcctaccaagaaaatatcagatcctttagcaggcgactgtaagtgcgtggagggttcgaagaagactagcagtcaggaatacgatgatcgtaaagttcaagcgggcatggagtttgagaacgcgttgcaaaactttatatttgttccaaacattcggaaaagcaagaatggatcgtctgacaaatcagacggagcggaaggtgcagctctcgattctaatgctattccaaatggaggagctactaacccttcacgtagaaggagagacgttgcgctcgagccagagctcgacgatgtagagggcagtgtacttctacgccatgtgcgctccatcacagacgataccgatgcatttttcgaaaaggacgacgaaaatacctataaagacgaagaagacttgtcctccaacaaacaattctatgaggtgtttgccaaggaattgccaccaaatcaaacacattttgtctttgaaaaactgcgccacttcacccgctacgctatcttcgtggtagcctgtagagaagaaatccccagcgaaaaattaagggacaccagttttaagaagtcgctctgcagcgattatgacaccgttttccaaactacaaagagaaagaaatttgccgacatagtcatggacctaaaagtagatttagaacacgccaacaacaccgagtccccagtacgggttcgctggacgccaccagtagatcccaacggagaaattgtcacctatgaagtggcctacaagttgcaaaaacccgatcaagtggaagaaaagaagtgcattccggctgctgacttcaaccagactgccggttatttaataaagctcaacgagggcctttacagcttcagggtgcgagccaattcaatagcgggatacggcgatttcacggaagtcgaacatataaaagttgagcctccgccgagctatgctaaggtctttttctggctactgggaatcggcctagcgttcctgatcgtttccctgttcggctatgtctgttacctgcacaagaggaaggttccctctaatgaccttcatatgaacacagaggtgaatccgttctatgcgagcatgcaatacatcccagacgattgggaggtgctgcgagagaacatcattcagttggctccactaggccagggatcctttggcatggtgtatgagggtatcctgaagtcctttccacccaatggcgtggatcgcgagtgtgccattaagactgtcaacgaaaatgctacggatcgcgagcgaaccaatttcctgagcgaggcgagcgtcatgaaggagttcgatacgtatcatgtcgtaagattgctcggtgtttgctccaggggtcagccggctctggtggtcatggagctaatgaagaagggtgatcttaagtcctatttgcgtgcccatcgtcccgaggagcgggatgaggccatgatgacgtatcttaatcgcatcggagtgactggtaatgtgcagcctcctacttatggaagaatctaccagatggccattgagattgcggatggcatggcatatttggccgccaagaagttcgtccatcgtgatcttgcagctcgaaattgcatggttgctgatgatttgacggtgaaaattggtgactttggaatgacccgtgacatctatgagacggattactatcggaagggcactaaagggctgctgccagttcgctggatgccaccggagagcttgcgagatggtgtctactctagtgccagtgatgtattcagctttggagtggttctctgggaaatggccaccttagcggctcagccataccagggactttccaacgagcaagtcctgcgttacgtcatcgatggcggtgttatggagaggccggaaaattgtcctgattttctgcataaactaatgcaaaggtgctggcatcataggtcttcggcgagacccagttttctggatatcattgcgtatctcgaaccacaatgccccaattcacaatttaaggaagtatccttctatcactcagaggcaggtctgcagcatcgggaaaaggagcgcaaggaacgcaatcagctagatgcattcgcggcagtccccttggatcaagatctgcaggatcgggaacagcaggaggatgctaccacacctttacgaatgggcgattatcagcagaactcctcgttggatcaaccgcccgaaagccccatcgccatggttcctgccatccggattcattgcgagcagtactcctga

SEQ ID NO: 17 - DNA, 2058 bp, Drosophila melanogaster
atgaacaaatactcggcatttatagtctgcatttcgctcgtgcttttatttacaaaaaaggatgtggggagccataatgtggactcaagaatatatggtttccagcaatcatcaggtatttgccatatttacaatggcaccatttgtcgcgatgtcttgagcaatgcccatgttttcgtatcccccaatctcaccatgaacgatttggaggagcgattaaaggcagcttatggagtaatcaaggaatccaaggatatgaacgcaaattgccgcatgtacgctttgcccagcttgtgtttcagttcaatgccaatttgccggactccagagcgcacgaatctcttgtacttcgccaacgtggccacaaatgccaagcaactgaagaacgtcagcattcgacggaagagaaccaagtccaaggacattaagaacataagcatattcaagaagaagtccaccatctacgaggatgtgttcagcacagacatatcgagtaaatacccaccaaccagagagtctgagaacctaaaacgcatttgccgcgaagagtgcgaacttctggagaacgagctgtgccagaaggaatatgccattgccaagcgacatcccgtcatcgggatggtgggtgtggaggattgccaaaagttgccgcagcacaaggactgcctatccttgggcatcaccatcgaggtggataagacggagaattgttactgggaggatggatcgacatatagaggagtggccaacgtctccgcatccggaaagccatgtttgcgatggtcatggctgatgaaggaaatctccgatttccctgaactcatcggtcagaattattgcagaaatcctggaagcgttgaaaatagtccttggtgttttgtggactcctcacgtgaacgcataatcgaactttgtgatattccaaaatgtgcggacaaaatatggattgccattgtcggaacgactgcagccattattctaatattcataattatatttgcgataatacttttcaaaaggagaacaatcatgcactatggaatgaggaatattcataatatcaacacacccagcgccgataaaaatatctacggaaattcgcagcttaataacgcacaagatgctggcaggggaaatctgggaaatctatccgatcacgttgctttgaactccaaacttatcgaaagaaatactctgctgaggataaaccattttacgctgcaggatgttgagtttctggaggagctgggcgaaggagcttttggaaaagtctacaagggacagctcctgcagccgaacaaaaccaccataacagttgccatcaaggcgttgaaggaaaacgcctcggtgaaaacgcagcaggactttaagcgcgaaatcgaactaatctcggatctaaagcatcagaatatagtgtgcatattgggcgtagtgctcaataaggagccctactgcatgctgttcgagtacatggccaatggtgatctgcacgaattcctaatctcaaactcacccaccgaaggcaagtcgctgtcgcagttggaattcctgcaaatagctctacaaatcagcgaaggaatgcagtatctgtcggcccatcattacgtacatcgcgacttggcagctcggaattgcctggtaaacgagggtctggttgtgaagatatccgattttggactatccagagacatttacagctcagattattatcgagttcagtcaaagtcgctattgcctgtaaggtggatgccctcggaatcgatattgtatggaaagtttacgaccgagagcgatgtttggtcctttggagtcgttctttgggaaatatacagctatggaatgcagccatactacggttttagcaatcaggaagtaatcaatctcatccgttcacggcaactgctctccgctccggaaaactgtcccactgctgtctactcgctaatgatcgagtgctggcatgagcagtcagtaaaacgtccaacattcacagatatttcgaaccgtctcaaaacttggcacgagggccactttaaggccagtaatccagaaatgtaa

SEQ ID NO: 18 - DNA, 1554 bp, Drosophila melanogaster
atgggtaactgcctcaccacacagaagggcgaacccgacaagcccgcagatcgaatcaagctggacgacccgcccaccatcggagtcggagtgggcgtgccacaaatccccatgccctcacacgccggacagccaccggagcagatacgtccggttccccagatcccggagagcgaaacggcaggtgccaacgccaagatttttgtcgccctctacgactacgacgcccgcaccgacgaggatttgagcttccgcaagggagagcacttggagatactgaatgacacgcagggtgactggtggctggcgcggagcaagaagacacgttcggaaggctacattccatccaattatgtggccaagttgaaatcaatcgaagcagaaccgtggtacttccgcaaaatcaaacgcattgaggctgagaaaaaacttctactgccagagaacgagcacggtgcatttttaattcgcgattccgaaagccgtcacaacgactactcgctatcagtgcgcgatggcgatacggttaagcattatcgcatcagacaattggacgaaggcggcttcttcatcgccaggcgcacgacattcagaacccttcaggagctggtggaacactattcgaaggactctgatggcctatgcgtcaacctctgcaagccgtgtgtccagatcgagaagcctgtaactgaggggctttcgcaccgcactcgcgatcagtgggagatcgacagaacgtctttgaaattcgtgcgcaaactgggctccggacagtttggcgatgtctgggagggattgtggaacaacacaacacctgtggcaattaaaactctgaaatctggtacaatggaccccaaggatttcttagcggaagcccagatcatgaagaaactgcgccacaccaagcttatacagttgtacgctgtctgcactgttgaggagcctatctatattatcacagagttaatgaagcacggttcactgttggaatatctccaagccattgcaggcaagggtcgtagccttaaaatgcaaactctgattgatatggcagcgcaaatagctgctggcatggcttacttggagtcccagaattatattcatagggatttagcggcgcgcaatgtactggtaggcgatggaaacatcgtcaaaatcgccgactttggtttagctaggctcatcaaggaggacgaatacgaggcgcgggtaggcgccagatttcccataaaatggaccgctccagaggctgctaactacagcaaattctcaataaaatcggatgtttggagctttggcattcttctcacagaactggtcacctacggacgcataccatatccaggcatgaccaacgctgaggtgctaacgcaagtggagcacggctatcgaatgccgcaacctcccaactgcgagccgcgcctgtatgagattatgctggaatgttggcacaaggaccccatgcgcagacccacgtttgagacgctacaatggaaactggaagacttctatacatctgatcagagcgactacaaagaggcgcaggcctactga

SEQ ID NO: 19 - DNA, 1779 bp, Drosophila melanogaster
atgatcaagtgcgccctgaacgaggtgggatgcgaggagctgccctccggttgcgacgatgacctcaccctggagcagaacttcatcgagaatggctataacaacgaacagcagagcaatagcaatcacagtgcctcacagtccacgataataacgagcacgatcaccaccaccataacgactacaactaccacgacgccgtccaaggaaaactcaagactgaaattcaaagtgcccaagatccagaagaaatcaaaggccatccgcaatacattccgctccaagttgctcaatttccagttgaagcgctccaagccgtgcaaacagtgcaccaagagacgtcgcatccatcccagcaaaagtgtctttgattttgccaaagagttcgaggtggaacaaccggctggttcggcggcggatgagcaattctgcaactgtccgccagctggtcaaaagcctgttaagccatccgtccaaatatccggccacaaagatcacccgttcgagtccagttctggagagctggacgagaactcggatcgggacatcgacaacgacgaggaggaggaggatagcgccagtgacgacgtgctcagcatgaaggatcactgctattgcgtgcccagcctggcggccagtatatcgctctccacaaatcgtccgctttacgaggaggaatggttccatggcgttctgccgcgcgaggaagtggttcgattgctgaataacgatggtgacttcctggtccgcgaaacgattcgaaacgaggagagccagattgtgctcagtgtctgttggaatggccataagcacttcattgtccagaccaccggagagggtaatttccggttcgagggaccaccatttgccagcatccaggagctgatcatgcatcagtatcactcggaattgccagtgaccgtgaaatcgggagccatactccgacgacccgtttgccgggagcgctgggagctgagcaacgatgatgtggtacttctggagaggattggtcggggaaactttggggatgtctacaaggccaaactgaagtccaccaaactggatgtggctgtcaaaacctgtcgaatgaccctgcccgacgaacagaagcgtaaattcctacaggaagggcgcatcctcaagcaatacgatcatccaaatatcgtaaaattgattggcatttgtgtgcagaagcagcccatcatgattgtcatggaattggtgctcggtggttcgcttttaacttatttacgcaagaactccaatggcctcaccactcgccaacaaatgggcatgtgcagagatgcggcggcaggcatgcgatatctggagtccaaaaactgcattcatcgcgatctggcggcgcgtaattgtctcgttgacttggagcacagtgtgaagatctccgatttcggaatgtctcgcgaggaagaggaatatatagtttccgatggcatgaaacaaatacctgtgaagtggacagctcccgaggccttgaatttcggcaagtacacttcgttgtgcgatgtgtggtcctatggcatactgatgtgggagatcttctccaagggcgacacaccctactccggcatgaccaactccagagccagagagcgcatcgatacgggatatcgtatgccaacgccgaagagcacgcccgaggagatgtaccgactgatgctccagtgctgggcagccgacgccgaatcccgaccgcatttcgatgagatctacaatgtggtggatgcactgattctgcgcctggacaacagccactaa

SEQ ID NO: 20 - DNA, 2685 bp, Caenorhabditis elegans
atgcaccatcccaaagaaacgcttcttatcgattcatctaatccttcttactcccacctcaccgagtaccgttttgataacctgaaacgtgaagagtctcgatcgacctcactttttggcgacaggagaagagtgatgaaaatcctgagtggattttccctcattattattgtcgttttcatatttgctacaagtcatgaacaggcgctctctaccactggagacctcacttcgagtactcagagtactacacatggaggtgttgtctttacatatccaactacaagaaaatctcccggtaaaggatgtgtcctgaattcgcagagatcaacgcctaaaaacttgaaacagtacactggaaacatttcagacgcttgtttagccggaataaaatcaagtaactgtaagacatggctaatgacaaatgcggtgattttgaaatactcagacgatgttgtcagcaattgcccttcgattttggaatttgtgaataaaacatcgttatcatgttcgggtaaaagtcagattcaatatatgtatcctcagagtgattctgcgtcaagtgattgcaatcactcttatgacttcaactcaaatgctctgaacagagcaatatataacttcaactacagcaagaccttaatctccacgtcatatgccaatactcctggattcgctatgtatacatttttgctgaagattatgaactgtgtcaacaaaaacggaataaaacttgacgccggaattctcaacatttttacggacatgacctatattgatttatgtgaaagtgatgttttcatgagctcgtttccagatactctgaacaagcttattgaggcggggtatattgtcaaattttatttcctgaatcaaaatttgcaagatactcaaaaaaacgttgaaaacgtactagctggatgtaaatacatgaattcaagatcgtactgcgaaattgtagactggagctatcattcggaaaatcctaatgagtttgaaatttgcatcccagattcacagcccagtgggaagaaagaagactttaattggcaacttcttctaattattggtataccttgtataagtttgacaatttgctgcattgcatttttcgtttgttgcttgaaatgtgctaaactgaaaatggcaatgatgagaatgaatgtattctcaaatgatactcaccaaaatcctgatgaaatggagctgaaaaagagatggatcgggatgagaaagaaattcaataaagatgttgagaatggaagttgtaaagagttaaacacccaaaaatggtctcacttcgcatcggcgaacaattacatggacatacaagcattggcaaatgctaataaaaaagatatatgggaaattgacacaaaaaatctgctcgtccaggaagaccatctccttggaaacggtgcatttgcaaacgtctataagggaatcgtaaaaggaaaaataccactactagttgtaaataatagtctcaacatgaccgtagaatcagaaaacaatggtcactatgaagctgccatcaagaagttaccagcccatgctgacgagcagaaccatttggattttttccatgaaattgattttatgaagcgtttgggccatcatccacatgtcatcagcatgttgggatgtgtgtcaaatccatatgagccattgatcgtggtggagtattgcgcacgtggtgacctgttgaagtttttgagaagacataaagattatgtgctgatgaatcgtgtacatattgaattatgtataagtatatacaagttcaaattaaaacttagaccgaacattgagatttcaaaaatcagtttccagaacaaaacagacgattgtccaattgaagcagacatgtgtctcagaatcaaagatttggtttctattgcttggcaagttgccgatggaatgtcatacctggcatcaaaaaactttattcaccgtgatttagctgcccgtaacattctgctcacaaaaagtttaactgcaaaggttagtgacttcggtctatgtcggtatatggattcagcactttataccgcaaaggggggccgtctccccatcaaatggatgtctgtagaagcattgaaactgtacgaattctccacaaaaactgatgtttggtcgtttggagtgttgttgttcgagattttctccatgggagatgttccgtatccaacaatacaacaagtagatatgctggaacaccttctcgctggtggccgcttgtcacagccattgaaatgtccgaatgagatatttaatatcatgcagaaatgttgggccgaaaagcctgaagacagaccagagtttaatgaaatgagaggagaaatcacagtgatgttgaacttggacgatgaaagttatggatatcttagcgtcgagtcacagggtggtccaaagtatacacaattaacaatgcaagattcaaaggaaacagctccatgctccactcctggaggatcacaagatatggacgaagacggggattatgatagtggctcagaaggccactcgcaaggaacttgtgctcagctcgaccaggttttgactgagagatttggtgaagaacagaagaaggaaatcaagcaaatcttttgtgagatcacttcgaaatcaatgcgaggcaaacgccgtcaatcgaattctacagtcagcacgtatcaatcttga

SEQ ID NO: 21 - DNA, 2376 bp, Caenorhabditis elegans
atgtttcaggagaatgcagtcaccaattgggaatgtcaaatcgaaagattcgtgatgagcaaatctcggcgtcttcgagtttcgacttgcaaagcactggacctcaacatgctcggtaagtggtttgggaactactcatttgaaattcgtactacagctcatcaagaatctggaagtggtgcctggtgtccgaagaatcaaataaactctctcagcaaagaatggttgcagatttcgttttccgtggatacagtaataacttctgtggagacccagggacgatttgacgacggacgtggaatggagtatgcgaccgcattcaaaattcagtactggcgaccttcgctaaacgcatgggcatcttataaagacgattttgagctagagacaattcctgctaataatgacacggagcacgcaatccggcgacatcttgaccgggcaatcatagcaagaagaatcagaattgttccagtttcaaattccaccagaactgtttgcatgagagttgaagttttcggatgcccatttgatgatagtctcgtgttttacaatgtcgatcaaggcgatttgcaatctggcatctcttatcacgacttttcctacgatggtaatctcgccaactctccacacttaaccggcggtattgggaagttatacgacggcgaagtgggaaaaaacaatgtatttgttaatcaccacaaatgggttggatggagacgtaaaagaaatggcaatgtgaagttggcatttgagttttccgaattgagaaatatatcagggattttgattcatacgtcgaacgagttcaaaaagagcgcaaaggcattttcctcggctactgtgctattttcgataaatggaaaagacttctcagacaccatcgtacacttcaataatccggaagataccgaatcagaggtacctcgatggataaggattccagtgaacaatcggattgccaaagttgcaaagattcgtcttaactttggaactgactccgactggctgttcatttctgaagtgaattttgaatcaaatcacacaaatattgagcttctcaatgatgacgtggttattcccgattcggtttcatatttctccgtaaccgagcacgatgacggaactagcatgtttgctttcattatcttcttcttcatgttcctcatcgtggcagtcattattctgacagttctctaccgtaaacgcgagtatcgtgtgaaagcatcgtctccatctccaaatgcgaaacgggaaattctgttgacaattgacggaaacaccatcaagcatcacgtttctccgtcaacctatcaaatggctcgcgataatcttcagaatgcgttgattgagaaaatgcccatgtcaccgattataagcgattacgctgaaccggacattagtgtttgctccgatgtcaccgccaacactccattgctctatggaattgatggtccatatgatacacagaagagaagcaaccctttgtcatctatggtaaaatactccgattatggagaggtttattgcacaacacttccggaaattgctcgagacaagttgatttgcgtgagcagaattgggcaaggagagtttggtgaagtcgatttgtgtcagcttgaaaaccgaaaagttgcggtcaaaaaacttcatggaatcagtcaagccgacgagttttcttttcatagagaaattcgagtattaggaagtctcaaacatccgaacgtagttgaagtcgtcggagtatgcactatacaaaaaccaatactctgtatcatggaatatatggaaaatggcgacttgaaatcctacattttgaaaaaccctactatacaaacctcccaatgcatctcaatttgcacacagcttgccgcaggacttgcctatttggaatcatgtaattttgtgcatagagatattgctgctcgaaattgccttgttgacggagaaggcaatgtaaaaattgccgatttcggaatggcccgatctctttattctcaagaatattacaaagttgagggaaagtttgtgctcccgattcgctggatggcatgggaagctttgctactcggcaaattttccactgccagtgatgtttggggattcggagttaccatgtgggagatcttctcgctgtgctccgaaaaaccatactccgatatgacagatgatgatgtggtggagaatcttcagagcatgagctctactggatcattaaagcaagttctttcccgaccaaggatgtgtccatcaaagttgtacaacgagcaaattcttccgtgctggaactatgagagcagtcgccgacccagtttcgagaacgtccatcttcacctccagtcattggtgcacacttctcctcatattcatttttaa

SEQ ID NO: 22 - DNA, 3390 bp, Mus musculus
atgggaatggcctgccttacaatgacagaaatggaggcaacctccacatctcctgtacatcagaatggtgatattcctggaagtgctaattctgtgaagcagatagagccagtccttcaagtgtatctgtaccattctcttgggcaagctgaaggagagtatctgaagtttccaagtggagagtatgttgcagaagaaatttgtgtggctgcttctaaagcttgtggtattacgcctgtgtatcataatatgtttgcgttaatgagtgaaaccgaaaggatctggtacccacccaatcatgtcttccacatagacgagtcaaccaggcatgacatactctacaggataaggttctacttccctcattggtactgtagtggcagcagcagaacctacagatacggagtgtcccgtggggctgaagctcctctgcttgatgactttgtcatgtcttacctttttgttcagtggcggcatgattttgtccacggatggataaaagtacctgtgactcatgaaactcaggaagagtgtcttgggatggcggtgttagacatgatgagaatagctaaggagaaagaccagactccactggctgtctataactctgtcagctacaagacattcttaccaaagtgcgttcgagcgaagatccaagactatcacattttaacccggaagcgaatcaggtacagatttcgcagattcattcagcaattcagtcaatgtaaagccactgccaggaacctaaaacttaagtatcttataaacctggaaaccctgcagtctgccttctacacagaacagtttgaagtaaaagaatctgcaagaggtccttcaggtgaggagatttttgcaaccattataataactggaaacggtggaattcagtggtcaagagggaaacataaggaaagtgagacactgacagaacaggacgtacagttatattgtgatttccctgatattattgatgtcagtattaagcaagcaaaccaggaatgctcaaatgaaagtagaattgtaactgtccataaacaagatggtaaagttttggagatagaacttagctcattaaaagaagccttgtcattcgtgtcattaattgacgggtattacagactaactgcggatgcgcaccattacctctgcaaagaggtggctcccccagctgtgctcgagaacatacacagcaactgccacggcccaatatcaatggattttgccattagcaaactaaagaaggcgggtaaccagactggactatatgtgctacgatgcagccctaaggacttcaacaaatactttctgacctttgctgttgagcgagaaaatgtcattgaatataaacactgtttgattacgaagaatgagaatggagaatacaacctcagcgggactaataggaacttcagtaaccttaaggaccttttgaattgctaccagatggaaactgtgcgctcagacagtatcatcttccagtttaccaaatgctgccccccaaagccaaaagataaatcaaaccttctcgtcttcagaacaaatggtatttctgatgttcagatctcaccaacattacagaggcataataatgtgaatcaaatggtgtttcacaaaatcaggaatgaagatttaatatttaatgaaagtcttggccaaggtacttttacaaaaatttttaaaggtgtaagaagagaagttggagattatggtcaactgcacaaaacggaagttcttttgaaagtcctagataaagcacataggaactattcagagtctttcttcgaagcagcaagcatgatgagtcagctttctcacaagcatttggttttgaattatggtgtctgtgtctgtggagaggagaacattctggttcaagaatttgtaaaatttggatcactggatacatacctgaagaagaacaaaaattccataaatatattatggaaacttggagtggctaagcagttggcatgggccatgcattttctagaagaaaaatcccttattcatgggaatgtgtgtgctaaaaatatcctgcttatcagagaagaagacaggagaacggggaacccacctttcatcaaacttagtgatcctggcattagcattacagttctaccgaaggacattcttcaggagagaataccatgggtacctcctgaatgcattgagaatcctaaaaatctcaatctggcaacagacaagtggagcttcgggaccactctgtgggagatctgcagtggaggagataagcccctgagtgctctggattctcaaagaaagctgcagttctatgaagataagcatcagcttcctgcacccaagtggacagagttagcaaaccttataaataattgcatggactatgagccagatttcaggcctgctttcagagctgtcatccgtgatcttaacagcctgtttactccagattatgaactactaacagaaaatgacatgctaccaaacatgagaataggtgccctagggttttctggtgcttttgaagacagggaccctacacagtttgaagagagacacttgaagtttctacagcagcttggcaaaggtaacttcgggagtgtggagatgtgccgctatgacccgctgcaggacaacactggcgaggtggtcgctgtgaagaaactccagcacagcactgaagagcacctccgagactttgagagggagatcgagatcctgaaatccttgcagcatgacaacatcgtcaagtacaagggagtgtgctacagtgcgggtcggcgcaacctaagattaattatggaatatttaccatatggaagtttacgagactatctccaaaaacataaagaacggatagatcacaaaaaacttcttcaatacacatctcagatatgcaagggcatggaatatcttggtacaaaaaggtatatccacagggacctggcaacaaggaacatattggtggaaaatgagaacagggttaaaataggagacttcggattaaccaaagtcttgccgcaggacaaagaatactacaaagtaaaggagccaggggaaagccccatattctggtacgcacctcaatccttgacggagagcaagttttctgtggcctcagatgtgtggagctttggagtggttctatacgaacttttcacatacatcgagaagagtaaaagtccacccgtggaatttatgcgaatgattggcaatgataaacaagggcaaatgattgtgttccatttgatagagctactgaagagcaacggaagattgccaaggccagaaggatgcccagatgagatttatgtgatcatgacagagtgctggaacaacaatgtgagccagcgtccctccttcagggacctttcgttcgggtggatcaaatgcgggacagtatag

SEQ ID NO: 23 - DNA, 3246 bp, Mus musculus
atggcacctccaagtgaggagacacctctgatccctcagcgctcttgcagcctctcatcctcagaggcaggagccctgcatgtgctccttcctccccggggacctgggcctccccagcgattgtcattctcttttggggactacttggctgaggatttatgtgtgcgagctgccaaggcctgtggcatcctgcctgtttatcattcgcttttcgctctggccactgaggacttctcttgctggtttcccccaagccacatcttctgcatagaggacgtggacactcaagtcttggtctacaggctacgcttttatttccctgactggtttgggctggagacatgtcaccgctttgggctgcgcaaagatttgaccagtgccatccttgacttacatgttttagaacatctctttgctcagcaccgcagtgacctggtgagtgggcgcctcccggtgggccttagcatgaaggagcagggagagttcctgagcctggccgtgctggacttggcccagatggctcgtgagcaggcccagcgcccaggagagctgctgaagacggtcagttacaaagcctgtctgccgcccagcctgcgcgatgtgatccagggccagaacttcgtgacacgcaggcgcatccgcaggaccgtggtcttggcgctgcgcgtgtggtcgcctgccaggccgaccgctacggctcatggccaagtatatctggacctggagcggctacatccagcggccaccaccgagaccttccgtgtggggctcccgggcgcccaggaggagccggggcttctgcgtgtggcgggggacaacggcatctcctggagctccggggaccaggagcttttccagaccttctgtgactttccggaaatcgtggatgtcagcatcaagcagcccacgtgtgggtccggcagggagcaccggctggtcactgtcaccaggatggacggccacatcctggaagcggagtttccggggctgcctgaggcgctgtctttcgtggccctcgtggatgggtacttccgcctgatctgcgactccaggcattatttctgcaaggaggtggcggcgccacggctgctggaggaggaggcggagctgtgccatggacccatcacgttagactttgccatccacaagctgaaggccgctgcgtccctcccaggcacctatattctccgccgcagcccgcaggactatgacagctttcttcttaccgcctgcgtccagactcctcttggccccgactacaagggctgcctcatccgccaggaccccagcggggctttctccctggttggcctcagcagccccacagaagcctgcgggacgtgcttgcagtgctggaattctgggctgcgagtagacggtgctgccctgaacctaacatcctgctgcgctcccagacccaaggaaaagtccaatttgatcgtggtgcgaaggggctgcacccccgcgcctgcccctggctgctccccgtcctgctgtgcgctgacacagctgagcttccacacaattccaacggacagcctgggacacgagaacctgggtcacggttcttttaccaagatcttccgtggccgcaggcgggaggtcgtggatggtgagacacatgactcggaagtcctcctgaaggtcatggactccagacatcggaactgcatggagtcttttctggaagccgcaagcttgatgagccaagtatcctacccgcacctggtgttactgcacggcgtctgcatggctggagacagcatcatggtgcaggaatttgtgtatctaggagcaattgacatgtacctgcgcaagcgtggccacctggtgtcagccagctggaaactgcaggtgaccaagcagctggcatatgcccttaactacttggaggacaaaggccttcctcacggcaacgtctcagcacggaaggtgctcctggctcgtgaggggggtgatgggaatccacctttcattaagctgagtgatcctggtgtcagtcccactgtgctgagcctggaaatgctcaccgacagaataccctgggtggcccccgaatgtctccaggaggctcagacactctgcttggaggctgacaagtggggctttggagccaccacgtgggaggtgttcagcgggggacccgcccacatcacctcgctggagcccgccaaaaagctgaagttctatgaggaccagggacagctgcccgctctcaaatggacagaactggcgggacttatcacacagtgcatggcgtatgatcctggccggcgcccctccttccgagctatcctcagagacctcaacggcctcattacatcagattacgagctcctctcagaccccacacctggcatcccgagtcctcgagatgagctgtgcggtggcgcccagctctatgcctgccaggaccccgccatattcgaggagagacaccttaagtacatctctttgctgggcaagggcaactttggcagcgtggagctgtgccgctatgaccccctggacaatacgggacccctggtggcagtgaaacagctacagcacagcgggccagaccagcagagggacttccagcgggagattcagatccttaaggctctgcacagcgacttcatcgtcaagtaccggggagtcagctatgggccaggtcgccagagcctgcggttggtgatggagtacctgcccagcggctgcctgcgagacttcctgcagcgccatcgcgcggccctgcacaccgaccgcctactgctgttcgcttggcagatctgcaagggcatggagtacctgggtgcgcgccgctgcgtacaccgtgacctggctgcgcgcaacatcttggtggagagcgaggctcatgtgaagatcgcggactttggcctcgctaagctgctgcccctgggaaaggactactacgtggtccgcgagcctggccaaagccccatcttttggtatgccccggagtccctatctgacaacatcttctcccgccaatctgacgtgtggagcttcggagtggtgttgtacgagctcttcacctactgcgacaagagctgcagcccatccgctgagttcctgcgcatgatggggcctgagcgtgaaggacccccgctctgccgcctcctggagctgctggcagagggccgacgcctcccaccacctcccacctgccccaccgaggttcaggagctcatgcagctgtgcgtggcgcccagccgcacgaccggccagccttcggcaccctga

SEQ ID NO: 24 - DNA, 1518 bp, Mus musculus
atggcaaggcgaagctcccgggtctcctggctggcctttgaaggctgggaatctagggacctgcctcgggtgagccctagattgttcggagcttggcaccccgcgcctgctgcagctaggatgccaacgcgctgggcccctgggactcaatgcatgaccaagtgtgagaactctcgccccaagcccggtgagctagcctttcgaaagggtgacatggtgaccatcttggaggcctgtgaggacaagagctggtaccgagccaagcaccatggcagtgggcaggaagggctgctggcggccgctgctctgcgacagcgggaggccctctccacagaccccaagctcagcctcatgccatggtttcatggcaagatctccggccaggaagccatacagcagctgcagccacccgaggacgggctgttccttgtgagggaatcagctcgtcaccctggagactatgtcttgtgtgtcagtttcggccgtgacgtcatccactaccgtgttttgcatcgagatgggcacctcaccatcgatgaggccgtgtgtttctgtaacctgatggacatggtggagcactacaccaaggacaagggggccatctgcaccaagctggtgaagccaaggaggaaacagggcgcaaagtctgcagaggaggagctcgccaaggctggctggctactcgacctgcagcatctgactctgggagcacagattggagagggggagtttggagccgtcctacagggtgagtacctgggacagaaggtggctgtgaagaatatcaagtgtgatgtgacagcccaggccttcctggatgagacggctgtgatgacgaagctgcagcacaggaacctagtgcgactcctgggtgtgatcctgcaccacggcttgtacattgtcatggagcacgtgagcaagggcaacctggtgaacttcctgcgcacgcggggccgtgctcttgtgagcacctctcagcttctgcagtttgctcttcatgttgctgaaggcatggaatacctggagagcaagaagctggtgcaccgggacctggctgctcggaacatcctggtctctgaggacttggtggccaaggtcagtgactttggcttagccaaggcagagcgcaaggggctggactcaagccggctgccagtcaagtggacggcacctgaggctctcaaaaacgggcggttctccagcaagtcggatgtctggagttttggggtgctgttgtgggaagtcttctcttatggaagagccccataccccaagatgtcgctaaaggaggtttcagaggctgtggagaagggttaccgcatggagccccccgatggctgcccaggctctgtgcacaccctcatgggtagctgctgggaggcagagcctgcgcgccgaccacccttccgcaaaatagtggagaagctgggccgtgagctccgcagtgtgggtgtctcggcccccgctgggggacaggaggctgagggctcagctcccacacggagccaggacccctga

SEQ ID NO: 25 - DNA, 1490 bp, Mus musculus
tggagcccttcctcaggaagcggctcactttcttgtcctttttctgggataagatatggccagcggatgaatcggaggaagacatccccaggatccagggacacgacgacaacccagtgccggagcaagccgctgccgttgaaccttgtagcttcccagccccacgcgcccgactcttccgcgcgctctacgacttcactgctcgatgtgcagaggaactgagcgtcagcggtggggacagactctacgccctcaaggaggagggggactacatctttgcccaaaggctctctggtccacccagcaccggactagttcctgtcacctaccttgccaaggctaccccggagccgccctcagaccaaccttggtacttcagtgggatcagcagggctcaggcccagcagttgctcttgtctcctgccaatgcaccaggggccttcctcatccggcccagcgaaagcagcatcgggggctattctctatcagtcagggcccaggccaaagtctgccactaccgcatctgcatggcacccagtggcagcctctatctgcaggagggccaactcttccccagcctggatgcactgctggcttactacaagaccaactggaagctgatccagaaccctctgctgcagccctgcataccccagatacccttggttcaggacgagtgggaacgaccacgttcagaatttgtcttcggaagaaagctgggtgaaggtttcttcggggaggtgtgggaaggcctgtggctgggctctatccctgtggcagtgaaggttatcaaatcagctgacatgaagctggcagacctcaccaaggagattgaggcactgaagagcttgaggcatgagaggctgatccggctgcacgctatatgttccctcggtgaacctgtgtacatcgttactgaactcatgggcaagggcaacttgcaagtctacctgggcagctctgagggaaaggccctgagcctgccccatctactgggatttgcctgccaggtagctgagggcatgagctacctggaggagcggcgtgtcgtccaccgggacttggctgccaggaacgtgctggtgggtgatgacctcacctgcaaggtagctgattttggcctggccagactgctcaaggatgatgtctactccccaagcagtggctccaagatccctgtcaagtggacggcacctgaggctgctaattaccgtgtcttttcccaaaagtcagatgtctggtcctttggcatcctgctgtatgaggtcttcacttatggccagtgtccctatgaaggaatgaccaaccatgagacgctacagcagattagtcgtggataccggctgccacgcccagctgtctgcccagcagaggtctatgtgctcatggtagagtgctggaagggcagccctgaggagcgtcccacctttgccatactgagggagaagctgaatgccataaacagacgcctccatctgggcctcacgtga